(19) United States
(12) Patent Application Publication
(10) Pub. No.: US 2009/0029949 A1
(43) Pub. Date: Jan. 29, 2009

(54) GPCR Ligands Identified by Computational Modeling

(76) Inventors: Abby L. Parrill-Baker, Memphis, TN (US); Gabor J. Tigyi, Memphis, TN (US); Donna Hope Perygin, Oakland, TN (US); James I. Fells, Memphis, TN (US)

Correspondence Address: DONNA J. RUSSELL, 1492 ANTHONY WAY, MT. JULIET, TN, 37122, US

(21) Appl. No.: 11/754,123

(22) Filed: May 25, 2007

Related U.S. Application Data
(60) Provisional application No. 60/808,398, filed on May 25, 2006.

(51) Int. Cl. A61K 31/19; A61K 31/197; A61K 31/66; A61K 31/4035; A61K 31/47; A61K 31/435; A61K 31/506; A61K 31/444; A61K 31/498; G06F 17/30

(52) U.S. Cl. 514/120; 514/563; 514/557; 514/567; 514/417; 514/311; 514/290; 514/252.14; 514/334; 514/562; 514/249; 707/5; 707/E17.014

(57) ABSTRACT

Disclosed are pharmacophores for developing and screening compounds having G-protein-coupled receptor antagonist activity, including LPA₁, LPA₂, LPA₃ and S1P antagonists. These compositions have therapeutic benefit in the fields of cancer chemotherapy, cardiovascular disease prevention, and fertility protective agents during radiation and chemotherapy.

### CROSS-REFERENCE TO RELATED APPLICATIONS

This application claims the benefit of priority of earlier-filed U.S. provisional patent application No. 60/808,398 filed May 25, 2006, which is incorporated herein by reference.

### STATEMENT OF GOVERNMENT RIGHTS

This invention was made with government support under USPHS HL61469, CA92160, awarded by the United States National Institutes of Health. The United States government has certain rights in the invention.

### FIELD OF THE INVENTION

The present invention relates to molecules affecting cell signaling through cellular receptors and methods for identifying those molecules. More specifically, the invention relates to compounds that act as agonists or antagonists of sphingosine-1-phosphate (S1P) receptors and lysophosphatidic acid (LPA) receptors and pharmacophores that can be used to identify those compounds.

### BACKGROUND OF THE INVENTION

Sphingosine 1-phosphate (S1P) and lysophosphatidic acid (LPA) are structurally and functionally related lysophospholipids (LPL) growth factors. S1P and LPA are separately recognized by distinct subsets of the G protein-coupled receptor (GPCR) family, S1P₁₋₅ and LPA₁₋₄. LPLs mediate their effects through these G-protein-coupled receptors (GPCRs), of which the most completely characterized are those encoded by the endothelial differentiation genes (Edgs). Edg-1, -3, and -5 recognizes and responds to S1P, and Edg-2 and -4 generally recognize and respond to LPA. The cellular effects of the LPLs may generally be categorized into two categories. One category comprises the growth-related activities of LPA and S1P, including stimulation of proliferation, prolongation of survival, prevention and suppression of apoptosis, and processes in differentiation. A second group of cellular effects of LPA and S1P includes functions dependent on the cytoskeleton such as shape changes, aggregation, adhesion, chemotaxis, contraction, and secretion.

Sphingosine 1-phosphate (S1P) is a naturally occurring sphingolipid mediator and also a second messenger with growth factor-like actions in almost every cell type (1-3). S1P plays fundamental physiological roles in vascular stabilization (4), heart development (5), lymphocyte homing (6) and cancer angiogenesis (7).

Given the important metabolic roles played by LPA and S1P and their receptors, these molecules and the pathways in which they participate make important candidates for therapeutic drug design. Development of receptor subtype-selective pharmacophores could aid rational drug design and lead optimization as well as identification of novel molecular scaffolds through in-silico searches of large chemical libraries. However, the lack of crystal structures of GPCR makes this significantly more difficult. What are needed are compositions for modulating LPA receptor- and S1P receptor-mediated pathways and methods for identifying and/or designing such compositions.

### SUMMARY OF THE INVENTION

The invention discloses pharmacophores describing activity at the lysophosphatidic acid (LPA) receptors, LPA₁₋₃. Such pharmacophores are described by Scheme I

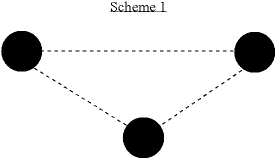

where the pharmacophore features may be described as follows:

A is an anionic functional group;

B and C are hydrophobic functional groups;

an LPA₁ Antagonist (A) has a distance between A and B of 7-11 Å, a distance between B and C of 6-10 Å, and a distance between A and C of 8-12 Å;

an LPA₁ Antagonist (B) has a distance between A and B of 7-11 Å, a distance between B and C of 5-8 Å, and a distance between A and C of 6-12 Å;

an LPA₁ Agonist has a distance between A and B of 15-17 Å, a distance between B and C of 9.2-11.2 Å, a distance between A and C of 15.5-17.5 Å;

an LPA₂ Antagonist has a distance between A and B of 5-9 Å, a distance between B and C of 4-7 Å, and a distance between A and C of 4-6 Å;

an LPA₂ Agonist (A) has a distance between A and B of 6-8 Å, a distance between B and C of 15.5-17.5 Å, and a distance between A and C of 18.5-20.5 Å;

an LPA₂ Agonist (B) has a distance between A and B of 10-12 Å, a distance between B and C of 12-14 Å, and a distance between A and C of 18.5-20.5 Å;

an LPA₃ Antagonist has a distance between A and B of 8-14 Å, a distance between B and C of 7-12 Å, and a distance between A and C of 12-16 Å;

an LPA₃ Agonist has a distance between A and B of 8.6-10 Å, a distance between B and C of 4.8-5, and a distance between A and C of 13.4-14.8;

anionic functional groups comprise phosphate, carboxylate, sulfate, sulfonamide, sulfite, nitro, tetrazole, phosphonamide, amide, hydroxy-oxazole and hydroxyl-thiazole; and

hydrophobic functional groups comprise saturated and unsaturated aliphatic and aromatic alkyl.

In some embodiments, aromatic alkyl comprises substituted or unsubstituted aromatic or heteroaromatic alkyl.

Also provided by the invention are pharmacophores that describe activity at the sphingosine 1-phosphate (S1P) receptors, S1P₁₋₅. An S1P₁₋₅ pharmacophore of the present invention may be described by Scheme 2

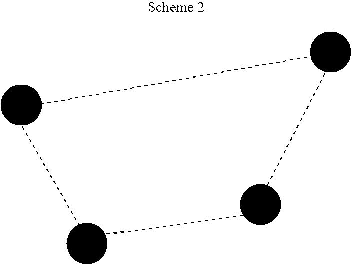

where the pharmacophore features may be described as follows:

A is an anionic functional group;

B is a cationic or hydrophobic functional group;

C and D are hydrophobic functional groups;

an S1P₁ Agonist has a distance between A and B of 5-7 Å, a distance between A and C of 10.5-11.8 Å, a distance between A and D of 13-16 Å, a distance between B and C of 5.5-7 Å, a distance between B and D of 9-9.5 Å, a distance between C and D of 4.5-5.5 Å, and B is a hydrophobic functional group;

an S1P₂ Agonist has a distance between A and B of 3-5.7 Å, a distance between A and C of 7.5-9.0 Å, a distance between A and D of 14.9-17.3 Å, a distance between B and C of 3.0-6.9 Å, a distance between B and D of 12.4-16.1 Å, and a distance between C and D of 10.3-12.0 Å;

an S1P₃ Antagonist has a distance between A and B of 2.4-3.3 Å, a distance between A and D of 6.1-8.4 Å, a distance between B and C of 2.4-6.1 Å, and a distance between C and D of 5.1-7.9 Å;

an S1P₄ Agonist has a distance between A and B of 3-4 Å, a distance between A and C of 9-10 Å, a distance between A and D of 17-20 Å, a distance between B and C of 9-10 Å, a distance between B and D of 16.5-18.5 Å, and a distance between C and D of 9-10 Å;

anionic functional groups comprise phosphate, carboxylate, sulfate, sulfonamide, sulfite, nitro, tetrazole, phosphonamide, amide, hydroxy-oxazole, hydroxyl-thiazole and trifluoromethyl;

hydrophobic functional groups comprise saturated and unsaturated aliphatic and aromatic alkyl groups; and

cationic functional groups comprise amine and guanidine functional groups optionally substituted by aromatic hydrogens on electron-deficient aromatic systems (i.e., those with nitro, trifluoromethyl and related substituents).

Hydrophobic functional groups comprising aromatic alkyl groups preferably comprise substituted or unsubstituted aromatic or heteroaromatic groups.

The invention also provides a method for identifying or distinguishing compounds having LPA receptor agonist, LPA receptor antagonist, S1P receptor agonist, or S1P receptor antagonist activity, the method comprising

providing the pharmacophore features and distances between features as described by the LPA receptor ligand pharmacophore of Scheme 1 and/or the S1P receptor ligand pharmacophore of Scheme II as input to a 3-dimensional database;

screening resultant matches (hits) by rigidly docking conformation matched to the pharmacophore into the receptor model; and

selecting structures for experimental screening based on their size and electronic complementarity to the receptor model.

The invention also provides compositions comprising LPA receptor agonists or antagonists having at least one anionic functional group comprising, for example, phosphate, carboxylate, sulfate, sulfonamide, sulfite, nitro, tetrazole, phosphonamide, amide, hydroxy-oxazole, hydroxyl-thiazole or trifluoromethyl, the anionic functional group being directly linked to a substituted or unsubstituted aromatic or heteroaromatic alkyl. In some embodiments, the direct link may be substituted for a molecular “spacer” comprising, for example, C₀₋₅ substituted or unsubstituted alkyl,

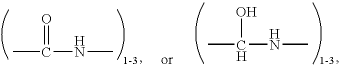

provided that the appropriate pharmacophore distance is maintained in the resulting molecule. In some embodiments, phosphate, carboxylate, or sulfate may be present as multiple anionic groups such as di- or triphosphate, for example. For each of the LPA receptor-specific or S1P receptor-specific classes of compounds described below, it is to be understood that the molecules may be described by the disclosed chemical structures and their corresponding pharmacophores.

The invention also provides a method of producing an LPA₁-specific response in a human or animal subject, the method comprising administering one or more LPA₁ receptor antagonists as in formula I

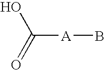

where

B is substituted or unsubstituted aromatic or heteroaromatic; and

A is either a direct link, C₀₋₅ substituted or unsubstituted alkyl,

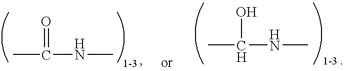

The invention also provides a method of producing an LPA₂-specific response in a human or animal subject, the method comprising administering one or more LPA₂ antagonists of formula I where

B is substituted or unsubstituted aromatic or heteroaromatic; and

A is a direct link or C₀₋₅ substituted or unsubstituted alkyl.

or one or more LPA₂ antagonists formula IIa or IIb

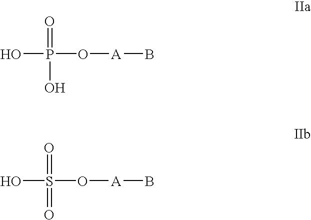

where

B is substituted or unsubstituted aromatic or heteroaromatic; and

A is either a direct link, C₀₋₅ substituted or unsubstituted alkyl,

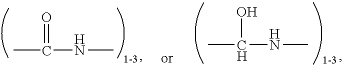

or combinations thereof.

The invention also provides a method of producing an LPA₃-specific response in a human or animal subject, the method comprising administering one or more LPA₃ agonists of formula I, IIa, or IIb where

B is substituted or unsubstituted aromatic or heteroaromatic;

A is a direct link, [CH₂]ₓ where x is 0-5,

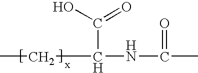

where x is 0-5,

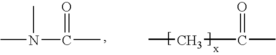

where x is 0-5,

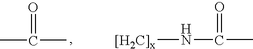

where x is 0-5,

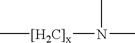

where x is 0-5, or

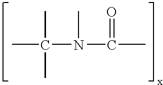

where x is 1-3; and

phosphate may be substituted with di- or tri-phosphate.

The invention also provides a method of producing an LPA₃-specific response in a human or animal subject, the method comprising administering one or more LPA₃ antagonists of formulas I, IIa, or IIb where

B is substituted or unsubstituted aromatic or heteroaromatic; and

A is a direct link, [CH₂]ₓ where x is 0-5

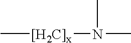

where x is 0-5,

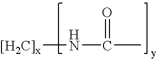

where x is 0-5 and y is 1-4, or

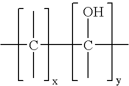

where x is 0-5 and y is 1-5.

Furthermore, the invention provides a method of producing an S1P₁-specific response in a human or animal subject, the method comprising administering one or more S1P₁ agonists of formulas I and IIa

where

A is a direct link; and

B is substituted or unsubstituted aromatic or heteroaromatic.

The invention also provides a method of producing an S1P₂-specific response in a human or animal subject, the method comprising administering one or more S1P₂ agonists of formulas I or IIa

where

A is

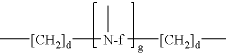

where d is 0-5, f is (CH₂)₀₋₅ or —C═O, and g is 1-5, or

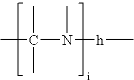

where h is —C═O or (CH₂)₀₋₅ and i is 1-5, and alkyl is optionally alkenyl; and

B is substituted or unsubstituted aromatic or heteroaromatic.

The invention also provides a method of producing an S1P₃-specific response in a human or animal subject, the method comprising administering one or more S1P₃ antagonists of formulas IIIa or IIIb

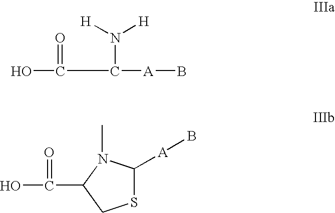

where

A is a direct link, [CH₂]ₓ where x is 0-5,

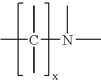

where x is 0-5,

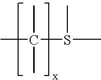

where x is 0-5 or -[CH₂]ₓ—S—[CH₂]ₓ— where x is 0-5 and

B is substituted or unsubstituted aromatic or heteroaromatic.

### BRIEF DESCRIPTION OF THE DRAWINGS

FIG. 1 shows chemical structures of representative LPA₁ antagonists identified using the LPA receptor agonist/antagonist pharmacophore.

FIG. 2 shows chemical structures of representative LPA₂ antagonists identified using the LPA receptor agonist/antagonist pharmacophore.

FIG. 3 shows chemical structures of representative LPA₃ antagonists identified using the LPA receptor agonist/antagonist pharmacophore.

FIG. 4 shows chemical structures of representative LPA₃ agonists identified using the LPA receptor agonist/antagonist pharmacophore.

FIG. 5 shows chemical structures of representative S1P₁ agonists identified using the S1P receptor agonist/antagonist pharmacophore.

FIG. 6 shows chemical structures of representative S1P₂ agonists identified using the S1P receptor agonist/antagonist pharmacophore.

FIG. 7 shows chemical structures of representative S1P₃ antagonists identified using the S1P receptor agonist/antagonist pharmacophore.

FIGS. 8a-8c is a series of graphs illustrating ligand-induced [³⁵S]GTPγS binding in S1P₁ mutants. Ligand-induced (0.1 nM-10 μM) GTPγS activation was calculated in transfected RH7777 cells. Activation dose-response curves of the mutants were normalized to WT S1P₁. A: The mutants displayed S1P-induced displayed three types of activations levels; no activation (F5.48Y-square), intermediate (L6.41-circle) and WT-like (T5.49G-triangle). B and C: GTPγS activation was carried in four S1P₁ mutants to characterized the ligand-induced activation by either S1P or SEW2871 (0.1 nM-10 μM).

FIG. 9a is an illustration of the S1P₁ agonist pharmacophore. Superposed structures of S1P and SEW2871 were derived by superposition of their complexes with the revised S1P₁ model. FIG. 9b illustrates the chemical structures of S1P (top) and SEW2871 (bottom).

FIG. 10 lists S1P₁ agonist hits from the NCI Database. Chemical structures of S1P, SEW2871, and hits identified in the Enhanced NCI Database Browser are shown. Panel A shows chemical structures of known S1P receptor agonists. Panel B shows chemical structures of good matches to the S1P/SEW2871 superposition. Panel C shows chemical structures of marginal matches to the S1P/SEW2871 superposition. Panel D shows chemical structures of negative matches to the S1P/SEW2871 superposition.

FIG. 11 is a graph of ligand-induced (0.1 nM-30 μM) GTPγS activation calculated in transfected RH7777 cells. Activation dose-response curves of the mutants were normalized to S1P.

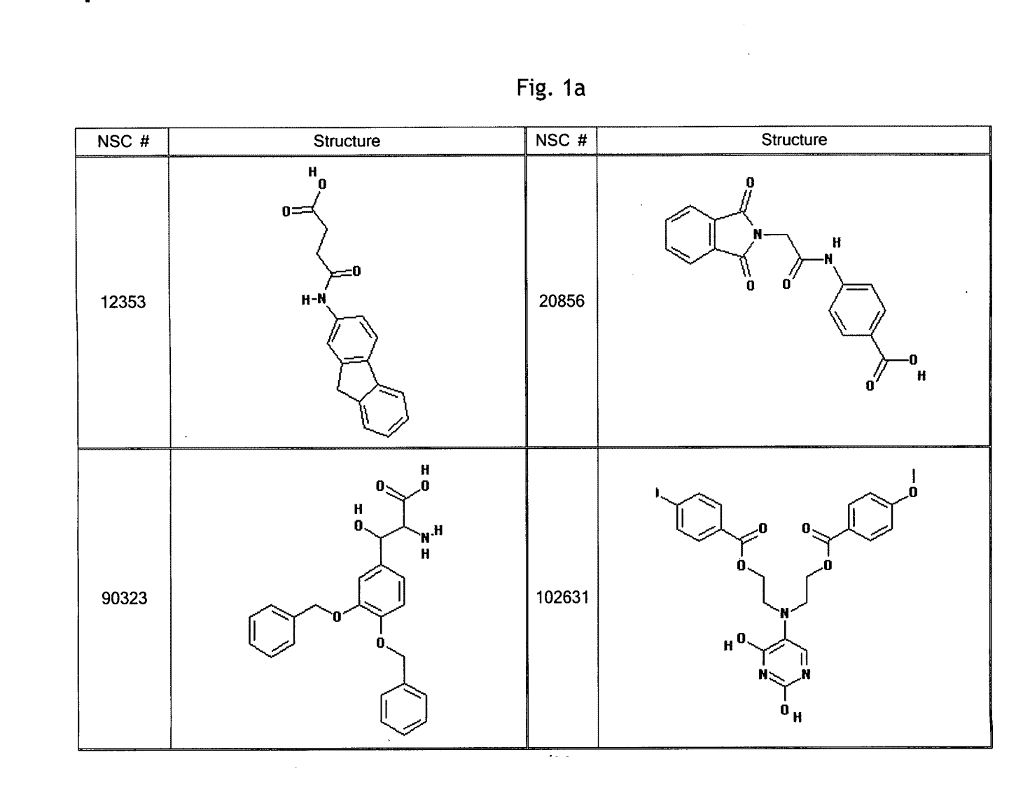

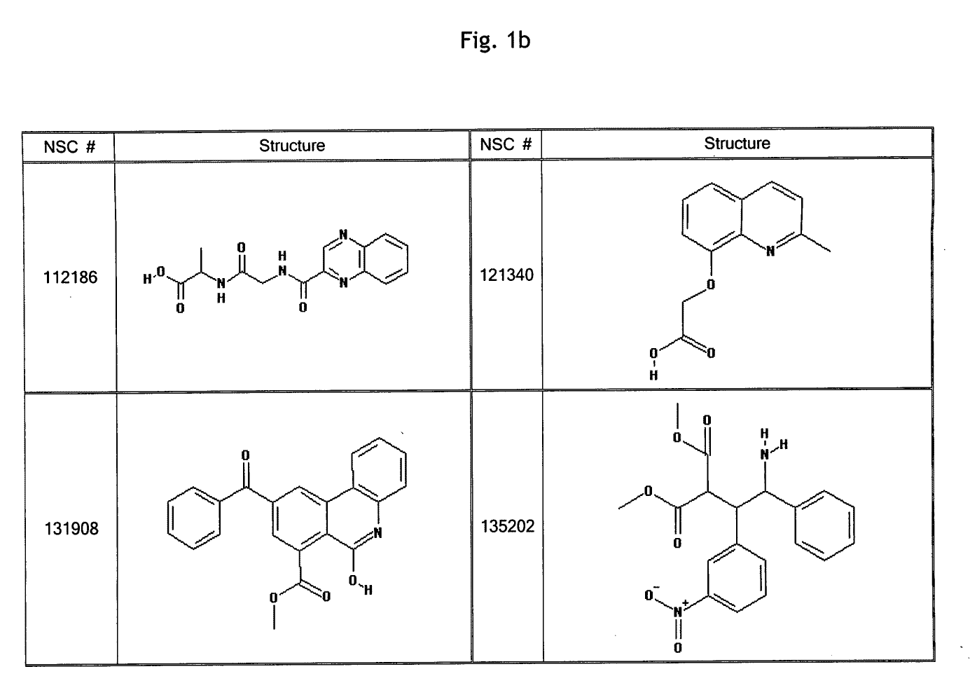

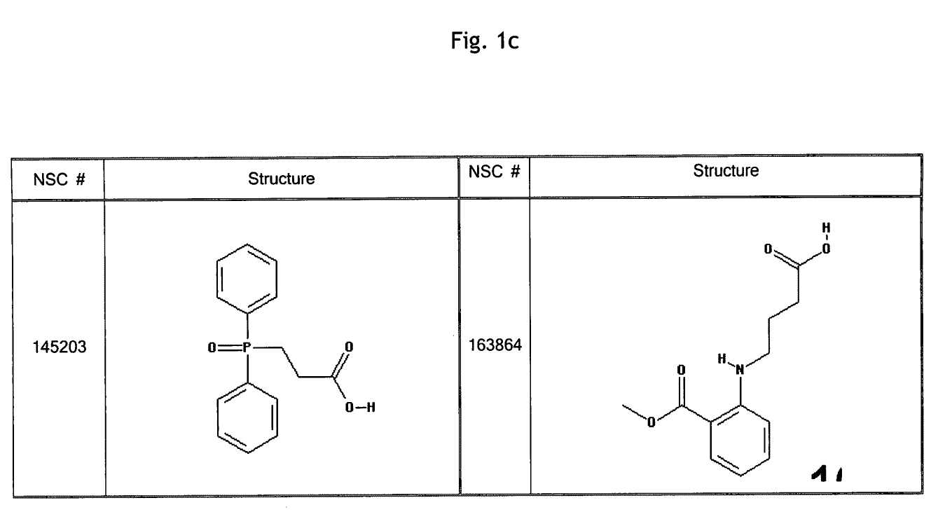

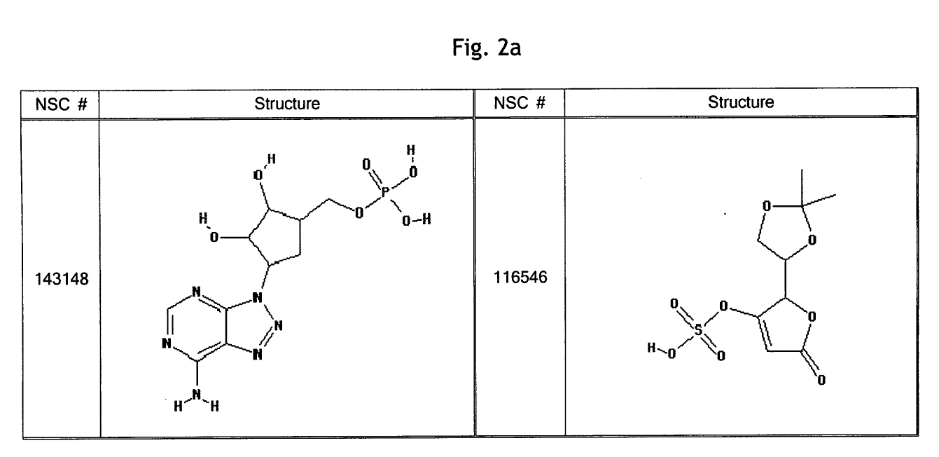

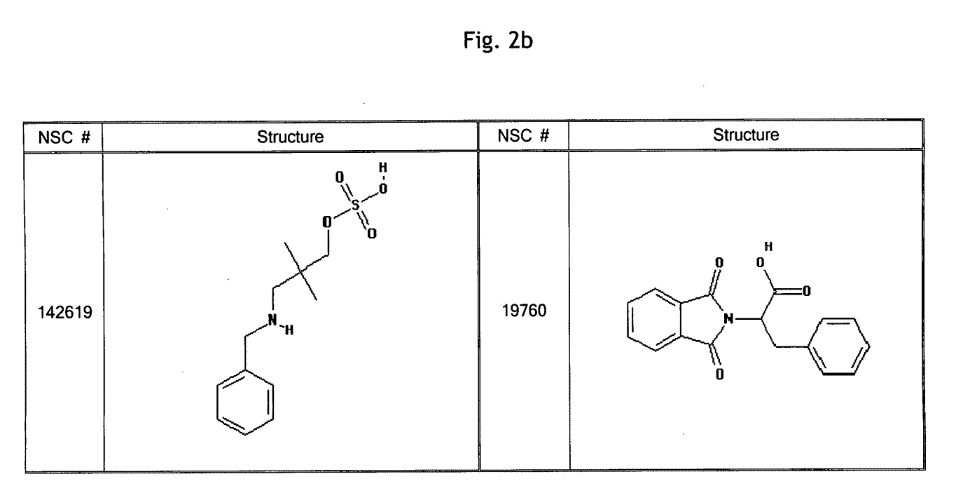

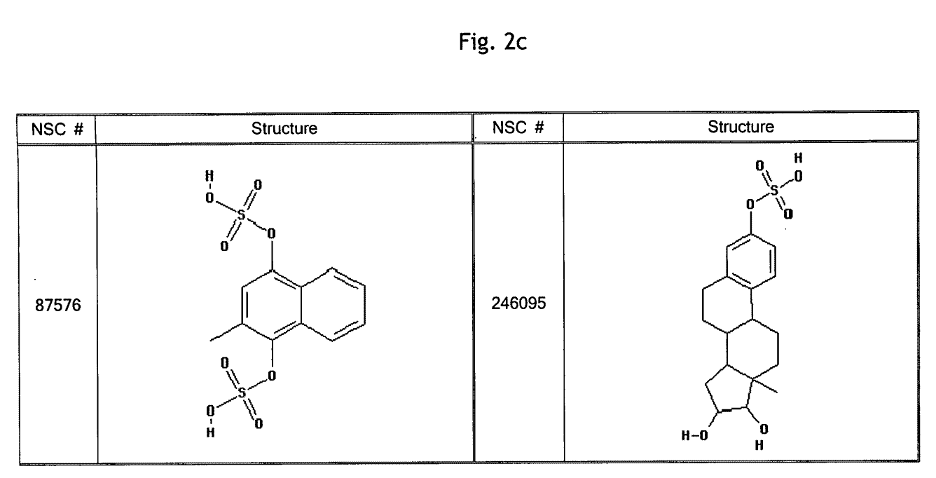

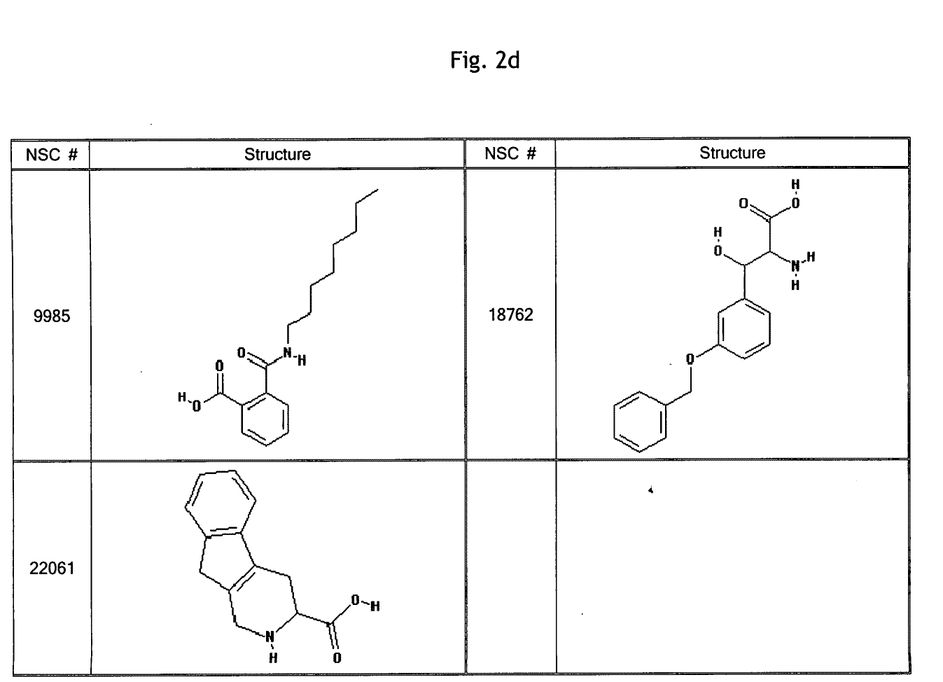

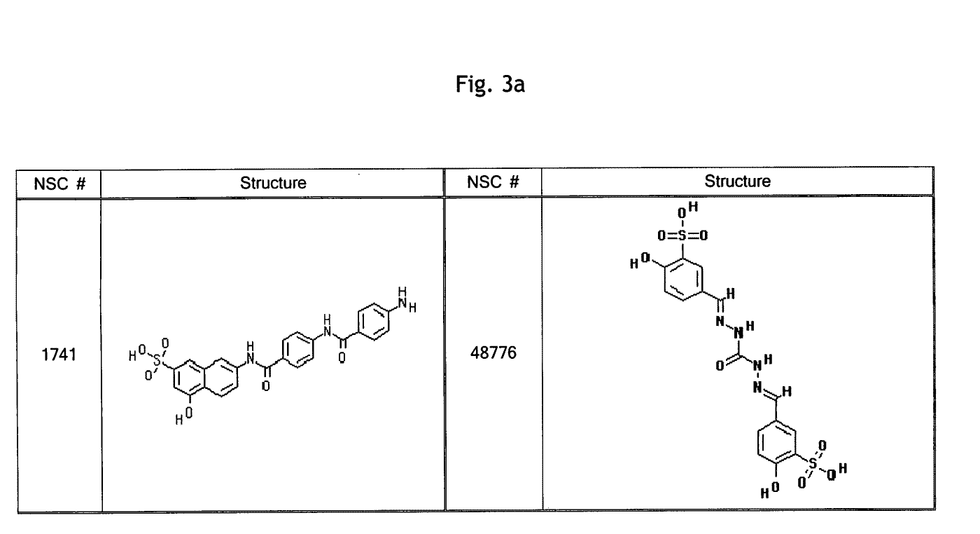

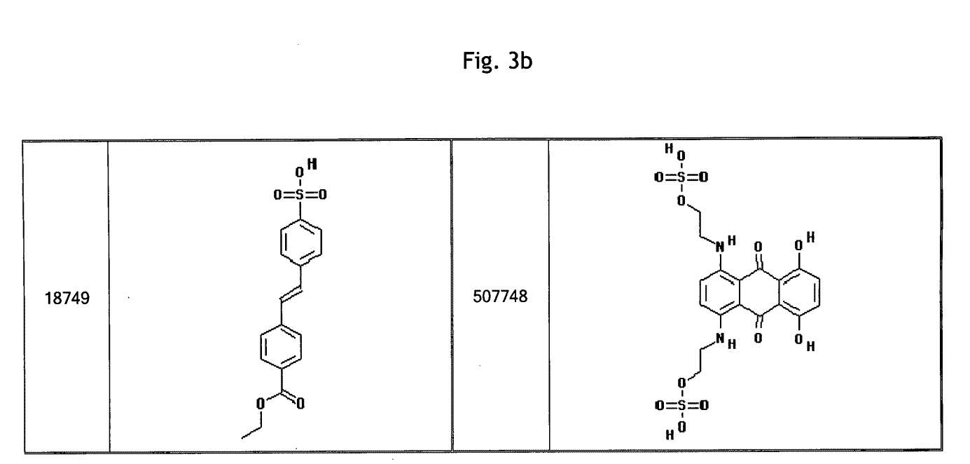

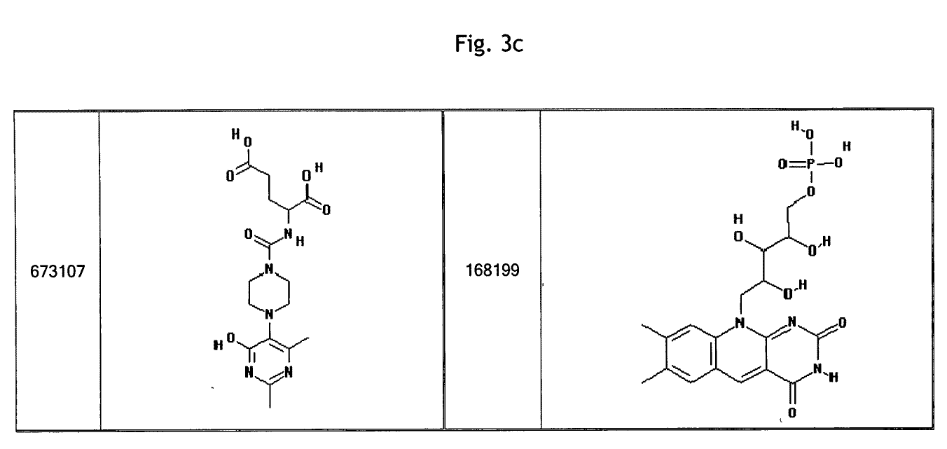

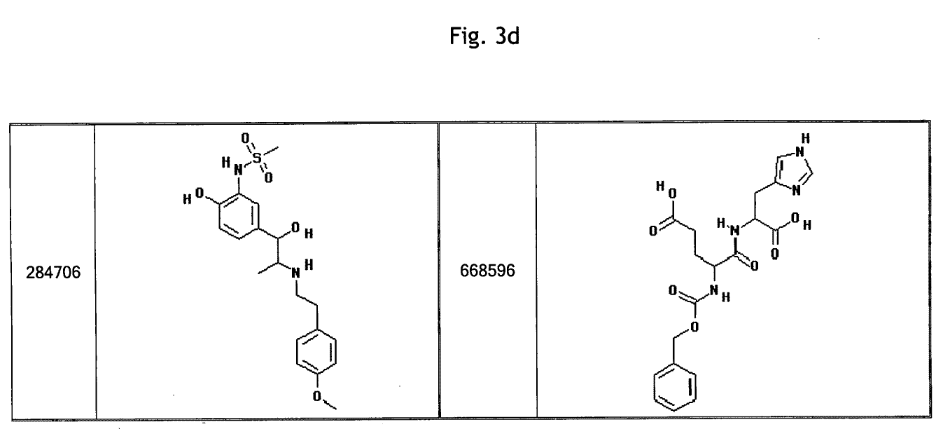

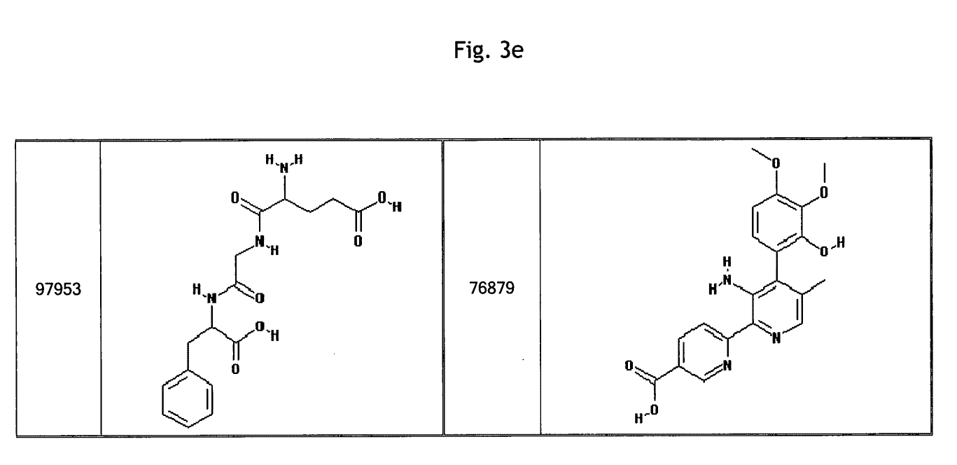

### DETAILED DESCRIPTION

The inventors have developed pharmacophores for screening compounds to assess their activity as LPA or S1P receptor agonists and antagonists. These pharmacophores have been successfully used by the inventors to screen compounds with generally unknown activity to identify those having agonist or antagonist activity for LPA₁, LPA₂, LPA₃, and S1P₁₋₃ receptors, providing a number of compounds described herein with specificity for the LPA₁, LPA₂, LPA₃, S1P₁, S1P₂, or S1P₃ receptors.

A pharmacophore is a geometric relationship among chemical functionalities (i.e., pharmacophore features) that produces a biological response. These pharmacophores have been used to mine chemical databases for novel structural scaffolds with potency reaching the low nanomolar range that have potential applications as cancer chemotherapeutics, cardiovascular disease preventatives, fertility treatments, and birth control agents. As used herein, a compound may be “described by” the pharmacophore or its features when its overall structure functionality corresponds to the given pharmacophore features.

The present invention provides pharmacophores describing activity at the lysophosphatidic acid (LPA) receptors, LPA₁₋₃. Such pharmacophores are described by Scheme I

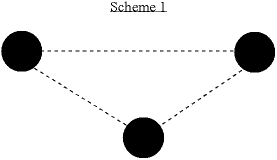

where the pharmacophore features may be described as follows:

A is an anionic functional group;

B and C are hydrophobic functional groups;

an LPA₁ Antagonist (A) has a distance between A and B of 7-11 Å, a distance between B and C of 6-10 Å, and a distance between A and C of 8-12 Å;

an LPA₁ Antagonist (B) has a distance between A and B of 7-11 Å, a distance between B and C of 5-8 Å, and a distance between A and C of 6-12 Å;

an LPA₁ Agonist has a distance between A and B of 15-17 Å, a distance between B and C of 9.2-11.2 Å, a distance between A and C of 15.5-17.5 Å;

an LPA₂ Antagonist has a distance between A and B of 5-9 Å, a distance between B and C of 4-7 Å, and a distance between A and C of 4-6 Å;

an LPA₂ Agonist (A) has a distance between A and B of 6-8 Å, a distance between B and C of 15.5-17.5 Å, and a distance between A and C of 18.5-20.5 Å;

an LPA₂ Agonist (B) has a distance between A and B of 10-12 Å, a distance between B and C of 12-14 Å, and a distance between A and C of 18.5-20.5 Å;

an LPA₃ Antagonist has a distance between A and B of 8-14 Å, a distance between B and C of 7-12 Å, and a distance between A and C of 12-16 Å;

an LPA₃ Agonist has a distance between A and B of 8.6-10 Å, a distance between B and C of 4.8-5, and a distance between A and C of 13.4-14.8;

anionic functional groups comprise phosphate, carboxylate, sulfate, sulfonamide, sulfite, nitro, tetrazole, phosphonamide, amide, hydroxy-oxazole and hydroxyl-thiazole; and

hydrophobic functional groups comprise saturated and unsaturated aliphatic and aromatic alkyl.

In some embodiments, aromatic alkyl comprises substituted or unsubstituted aromatic or heteroaromatic alkyl.

Listed in Table 1 are the distances between the pharmacophore features for each type of activity at the LPA receptors. In the case of LPA₂ agonism, for example, two pharmacophores are presented that differ in the position of hydrophobic point B by 4.7 Å.

TABLE 1
Distances Between Pharmacophore Features
Conferring LPA Receptor Activity

| | A-B Distance | B-C Distance | A-C Distance |
|---|---|---|---|
| Activity | (Å) | (Å) | (Å) |
| LPA₁ Antagonist (A) | 7-11 | 6-10 | 8-12 |
| LPA₁ Antagonist (B) | 7-11 | 5-8 | 6-12 |
| LPA₁ Agonist | 15-17 | 9.2-11.2 | 15.5-17.5 |
| LPA₂ Antagonist | 5-9 | 4-7 | 4-6 |
| LPA₂ Agonist (A) | 6-8 | 15.5-17.5 | 18.5-20.5 |
| LPA₂ Agonist (B) | 10-12 | 12-14 | 18.5-20.5 |
| LPA₃ Antagonist | 8-14 | 7-12 | 12-16 |
| LPA₃ Agonist | 8.6-10 | 4.8-5 | 13.4-14.8 |

Table 2 lists several examples of compounds screened and identified as LPA agonists or antagonists using the LPA agonist/antagonist pharmacophore of the present invention.

TABLE 2
Leads Identified Using the LPA Agonist/Antagonist Pharmacophores

| NSC # | Formula | Structure | LPA₁ | LPA₂ | LPA₃ |
|---|---|---|---|---|---|
| 143108 | C₁₈H₂₄N₅O₈P(M.W. 469.4) | 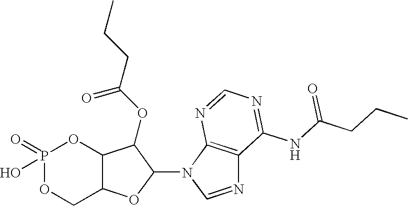 | Weak agonist | No Effect | IC₅₀ = 1309nM 61.0%inhibition of200 nM LPAresponse at 30JM |
| 77541 | C₁₉H₁₆ClNO₄(M.W. 357.8) | 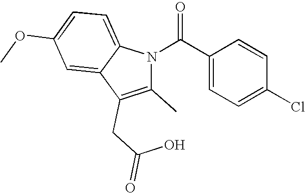 | Weak agonist | No Effect | IC₅₀ = 909 nM76.2%inhibition of200 nM LPAresponse at 30JM |
| 10188 | C₁₇H₁₅NO₅(M.W. 313.3) | 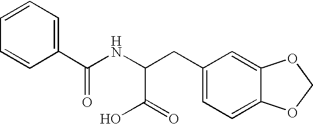 | Weak agonist | IC₅₀ = 6900nM (55.8%inhibition of200 nM LPAresponse at 30JM) | No effect |
| 3066 | C₁₈H₁₈N₄O₆(M.W. 386.4) | 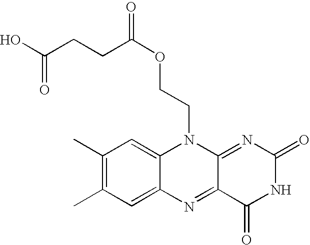 | IC₅₀: 197 nMKᵢ: 91.7 nM | IC₅₀: 167 nMKᵢ: 78.7 nM | IC₅₀: 103 nMKᵢ: 44.8 nM |
| 10167 | C₁₃H₁₆O₆(M.W. 268.3) | 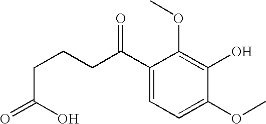 | No Effect | IC₅₀: 88 nM(47.4%Inhibition of 10nM LPAresponse) | IC₅₀: 517 nM(49.9%Inhibition of200 nM LPAresponse) |
| 16540 | C₁₄H₁₃NO₆(M.W. 291.3) | 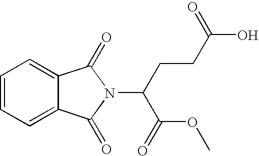 | No effect | IC₅₀: 100 nM(50.9%Inhibition of 10nM LPAresponse) | IC₅₀: 231 nM(59.7%Inhibition of200 nM LPAresponse) |
| 18760 | C₂₂H₂₄N₂O₇(M.W. 428.4) | 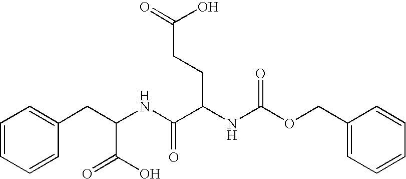 | No effect | IC₅₀: 20 nM(53.3%Inhibition of 10nM LPAresponse) | IC₅₀: 9 nM(58.7%Inhibition of200 nM LPAresponse) |
| 47091 | C₁₅H₁₂N₂O₈(M.W. 348.3) | 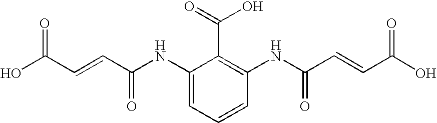 | No effect | IC₅₀: 355 nM(53.5%Inhibition of 10nM LPAresponse) | IC₅₀: 30 nM(81.7%Inhibition of200 nM LPAresponse) |
| 62586 | C₁₁H₁₃N₅O₄S(M.W. 311.3) | 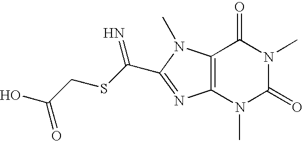 | IC₅₀: 234 nMKᵢ: 91.7 nM | IC₅₀: 172 nMKᵢ: 117 nM | IC₅₀: 35 nMKᵢ: 15.7 nM |
| 161613 | C₁₆H₁₉N₅O₁₀S(M.W. 473.4) | 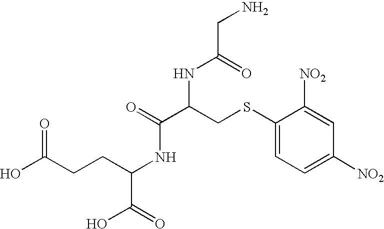 | No effect | No effect | IC₅₀: 24 nM(69.8%Inhibition of200 nM LPAresponse) |
| 255523 | C₁₇H₂₀N₅O₇P(M.W. 437.4) | 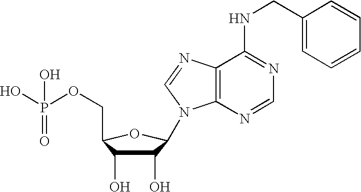 | No effect | No effect | PotentiatesLPA-inducedactivation |
| — | C₁₇H₂₀N₅O₆PS(M.W. 453.4) | 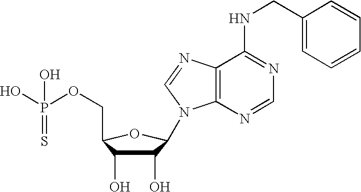 | No effect | PotentiatesLPA-inducedactivation | No effect |

Also provided by the invention are pharmacophores that describe activity at the sphingosine 1-phosphate (S1P) receptors, S1P₁₋₅. The inventors are using these pharmacophores to mine chemical databases for novel structural scaffolds that have potential applications as cancer chemotherapeutics, cardiovascular disease preventatives, and protective agents against cellular damage resulting from radiation and chemotherapy. An S1P₁₋₅ pharmacophore of the present invention may be described by Scheme 2

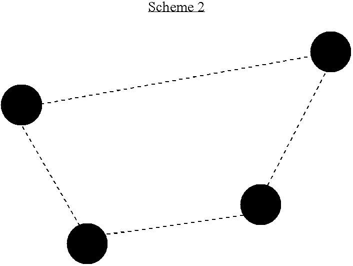

where the pharmacophore features may be describe as follows:

A is an anionic functional group;

B is a cationic or hydrophobic functional group;

C and D are hydrophobic functional groups;

an S1P₁ Agonist has a distance between A and B of 5-7 Å, a distance between A and C of 10.5-11.8 Å, a distance between A and D of 13-16 Å, a distance between B and C of 5.5-7 Å, a distance between B and D of 9-9.5 Å, a distance between C and D of 4.5-5.5 Å, and B is a hydrophobic functional group;

an S1P₂ Agonist has a distance between A and B of 3-5.7 Å, a distance between A and C of 7.5-9.0 Å, a distance between A and D of 14.9-17.3 Å, a distance between B and C of 3.0-6.9 Å, a distance between B and D of 12.4-16.1 Å, and a distance between C and D of 10.3-12.0 Å;

an S1P₃ Antagonist has a distance between A and B of 2.4-3.3 Å, a distance between A and D of 6.1-8.4 Å, a distance between B and C of 2.4-6.1 Å, and a distance between C and D of 5.1-7.9 Å;

an S1P₄ Agonist has a distance between A and B of 3-4 Å, a distance between A and C of 9-10 Å, a distance between A and D of 17-20 Å, a distance between B and C of 9-10 Å, a distance between B and D of 16.5-18.5 Å, and a distance between C and D of 9-10 Å;

anionic functional groups comprise phosphate, carboxylate, sulfate, sulfonamide, sulfite, nitro, tetrazole, phosphonamide, amide, hydroxy-oxazole, hydroxyl-thiazole and trifluoromethyl;

hydrophobic functional groups comprise saturated and unsaturated aliphatic and aromatic alkyl groups; and

cationic functional groups comprise amine and guanidine functional groups optionally substituted by aromatic hydrogens on electron-deficient aromatic systems (i.e., those with nitro, trifluoromethyl and related substituents).

Hydrophobic functional groups comprising aromatic alkyl groups preferably comprise substituted or unsubstituted aromatic or heteroaromatic groups.

TABLE 3
Distances Between Pharmacophore Features Conferring S1P Receptor Activity

| | Distances (Å) | | | | | |
|---|---|---|---|---|---|---|
| Activity | A-B | A-C | A-D | B-C | B-D | C-D |
| S1P₁ Agonist* | 5-7 | 10.5-11.8 | 13-16 | 5.5-7 | 9-9.5 | 4.5-5.5 |
| S1P₂ Agonist | 3-5.7 | 7.5-9.0 | 14.9-17.3 | 3.0-6.9 | 12.4-16.1 | 10.3-12.0 |
| S1P₃ Antagonist | 2.4-3.3 | | 6.1-8.4 | 2.4-6.1 | | 5.1-7.9 |
| S1P₄ Agonist | 3-4 | 9-10 | 17-20 | 9-10 | 16.5-18.5 | 9-10 |

*Feature B is a hydrophobic functional group

The invention also provides a method for utilizing a pharmacophore of Scheme I or Scheme 2 to develop and/or identify compounds having LPA receptor agonist or agonist activity, or S1P agonist or antagonist activity, the method comprising

providing the pharmacophore features and distances between features as described by the LPA receptor pharmacophore and/or the S1P receptor pharmacophore described herein as input to a 3-dimensional database;

screening resultant matches (hits) by rigidly docking conformation matched to the pharmacophore into the receptor model; and

selecting structures for experimental screening based on their size and electronic complementarity to the receptor model. Methods for computational analysis of chemical compounds using pharmacophores are described, for example, in the Textbook of Drug Design and Discovery, 3^{rd} ed. (Krogsgaard-Larsen, P. et al, eds., Taylor and Francis publishing, New York, N.Y. USA) and The Organic Chemistry of Drug Design and Drug Action, 2^{nd} ed. (Silverman, Richard, Elsevier Publishing, New York, N.Y. USA). Given the pharmacophores described herein, the practice of the method for using the pharmacophores to screen corresponding chemical compounds is well within the skill of those in the art.

Previously, the inventors and others had identified LPA₁₋₃ receptor agonists and antagonists having structural similarities with LPA, particularly in the presence of the phosphate head group and the acyl chain. Work published by Jalink, et al. (Biochem. J. (1995) 307: 609-616) indicated that, particularly for agonist activity, the acyl chain is an important element of the LPA molecule and modifications to the acyl chain affected agonist/antagonist activity. LPA receptor antagonists VPC 32183, VPC 32179, and VPC 12249, for example, sold by Avanti Polar Lipids, possess the acyl chain. Compounds cited for use by Kim et al., (WO 2004/052375), in a method comprising administering LPA or derivatives thereof to decrease neutrophil accumulation, contain an acyl chain. U.S. Pat. No. 7,169,818 (Lynch et al.) also describes LPA receptor agonists and antagonists having an acyl chain. The inventors, however, using the LPA pharmacophore of the present invention, have found that LPA receptor agonists and antagonists can comprise molecules lacking the acyl chain characteristic of lysophosphatidic acid. More specifically, compounds identified to be useful as LPA receptor agonists or antagonists using the pharmacophore of the invention include compounds having at least one anionic functional group such as, for example, phosphate, carboxylate, or sulfate, the anionic functional group being directly linked to a substituted or unsubstituted aromatic or heteroaromatic alkyl. In some embodiments, the direct link may be substituted for a molecular “spacer” comprising, for example, C₀₋₅ substituted or unsubstituted alkyl,

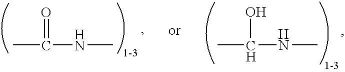

provided that the appropriate pharmacophore distance is maintained in the resulting molecule. For each of the LPA receptor-specific or S1P receptor-specific classes of compounds described below, it is to be understood that the molecules may be described by the disclosed chemical structures and their corresponding pharmacophores.

Compounds of the present invention therefore include compounds that are LPA₁ receptor antagonists as in formula I

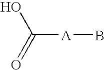

where

B is substituted or unsubstituted aromatic or heteroaromatic; and

A is either a direct link, C₀₋₅ substituted or unsubstituted alkyl,

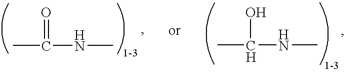

LPA₂ antagonists described by the present invention include those compounds of formula I where

B is substituted or unsubstituted aromatic or heteroaromatic; and

A is a direct link or C₀₋₅ substituted or unsubstituted alkyl.

LPA₂ antagonists described by the present invention also include those compounds of formula IIa or IIb

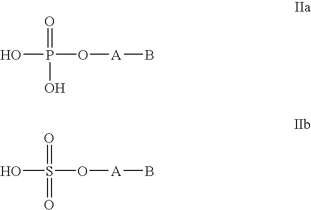

where

B is substituted or unsubstituted aromatic or heteroaromatic; and

A is A is either a direct link, C₀₋₅ substituted or unsubstituted alkyl,

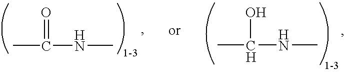

LPA₃ agonists identified by the pharmacophore of the present invention include compounds of formula I, IIa, or IIb where

B is substituted or unsubstituted aromatic or heteroaromatic;

A is a direct link, [CH₂]ₓ where x is 0-5,

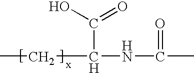

where x is 0-5,

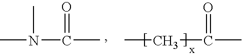

where x is 0-5,

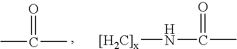

where x is 0-5,

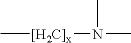

where x is 0-5, or

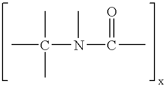

where x is 1-3; and

phosphate may be substituted with di- or tri-phosphate.

LPA₃ antagonists identified by the pharmacophore of the present invention include compounds of formulas I, IIa, and IIb where

B is substituted or unsubstituted aromatic or heteroaromatic; and

A is a direct link, [CH₂]ₓ where x is 0-5

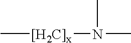

where x is 0-5,

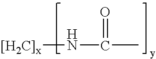

where x is 0-5 and y is 1-4, or

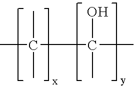

where x is 0-5 and y is 1-5.

S1P₂ agonists include compositions comprising compounds of formulas I or IIa where

A is

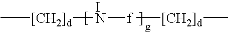

where d is 0-5, f is (CH₂)₀₋₅ or —C═O, and g is 1-5, or

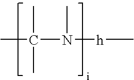

or where h is —C═O or (CH₂)₀₋₅ and i is 1-5, and alkyl is optionally alkenyl; and

B is substituted or unsubstituted aromatic or heteroaromatic.

S1P₃ antagonists include compounds of formulas IIIa and IIIb

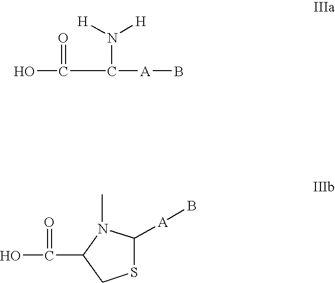

where

A is a direct link, [CH₂]ₓ where x is 0-5,

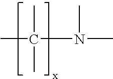

where x is 0-5,

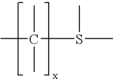

where x is 0-5 or -[CH₂]ₓ—S—[CH₂]ₓ— where x is 0-5 and

B is substituted or unsubstituted aromatic or heteroaromatic.

The invention therefore also provides a method for producing an LPA-receptor-specific or S1P-receptor-specific response in a human or animal subject, the method comprising selecting a compound for its LPA- or S1P-receptor specificity as an agonist or antagonist and administering such a selected compound to achieve a desired LPA-receptor agonist/antagonist-specific or S1P-receptor agonist/antagonist-specific result. In various embodiments, the method comprises administering compounds as described above for their receptor-specific activity. It is to be understood that the anionic functional groups provided for each receptor-specific class of compounds may be substituted by one of skill in the art by other anionic functional groups to achieve a molecule with similar functionality, these anionic groups including but not limited to phosphate, carboxylate, sulfate, sulfonamide, sulfite, nitro, tetrazole, phosphonamide, amide, hydroxy-oxazole, hydroxyl-thiazole and trifluoromethyl, for example.

Compounds identified by the method may have a variety of therapeutic uses, given the significant role of LPA, S1P, and their receptors in the mammalian body. Once synthesized, identified, or screened using the method of the invention, such compounds may be provided for therapeutic use via a variety of delivery routes such as, but not limited to, oral, nasal, intraperitoneal, intravenous, subcutaneous, and intramuscular. Administration may be provided as a single dosage, multiple dosages delivered at intervals over time, or modified release dosages for delivery of a single or multiple dosages as needed or over a period of time following initial administration, such as may be provided by a medication depot, pump, or other device.

The inventors had identified three basic amino acids, R3.28, K5.38, and R7.34 in S1P₁ and S1P₄ that form salt bridges with the phosphate group of S1P and are essential for ligand binding in one or both receptors (26, 27). They also pinpointed position 3.29, conserved as glutamine in LPA- and glutamate in S1P-specific members of the EDG family, as the single locus that determines ligand specificity for S1P versus LPA through its ion pairing with the ammonium moiety of S1P (28). The Q/N3.29 residue also plays an essential role in ligand binding because substitution to alanine results in a loss of S1P and LPA binding and receptor activation. They also elucidated differences between S1P₁ and S1P₄, as in the latter subtype K5.38 and W4.64 together compensate for the lack of a cationic residue at position 7.34 as in S1P₁ (27). These polar headgroup interactions are essential for ligand binding, activation, and specificity. However, the hydrophobic tail constituting the bulk of S1P has not been assigned a function and its interaction with the ligand binding pocket has not been elucidated.

The inventors experimentally validated a computational model of the ligand binding pocket of the S1P₁ GPCR surrounding the aliphatic portion of S1P. Mutagenesis-based validation confirmed 18 residues lining the hydrophobic ligand binding pocket, which the inventors combined with previously validated three head-group interacting residues to complete mapping of the S1P ligand recognition site. The validated ligand binding pocket provided a pharmacophore model, which was used for in-silico screening of the United States National Cancer Institute (NCI) Developmental Therapeutics chemical library, leading to the identification of two novel non-lipid agonists of S1P₁.

A computational model of S1P docked in the S1P₁ receptor was developed and the hydrophobic region of the ligand binding pocket has been experimentally validated with a “hit-rate” of 90%, in which mutations of 18 out of 20 residues predicted to interact with the hydrophobic tail displayed impaired or altered S1P-induced activation. Computational modeling was used to guide the mutagenesis strategy to gain insight into the structure-function relationship of S1P₁. The choice of replacement of residues in the predicted hydrophobic ligand binding pocket determined the type of effect observed in ligand-induced activation. For example, at least one of the two types of replacements introduced into four residues had little or no impact on Eₘₐₓ and only slightly increased the EC₅₀ values relative to WT. At the same time, at least one of the two replacements for four of these same residues had a major impact on Eₘₐₓ and/or EC₅₀. This established the refined nature of the computational predictions and at the same time provided the inventors with internal controls in a sense that receptor function was not always affected.

The experimentally validated predictions of the theoretical model localize the hydrophobic binding pocket to the transmembrane (TM) TM3, TM5, and TM6 domains. All but one of these residues are conserved in the EDG family of receptors. Of the 20 residues tested, three out of five in TM3, three out of ten in TM5 and two out of five in TM6 are identical in all EDG family S1P receptors. Of the eight identical residues among S1P receptors, five are also identical in the LPA receptors of the EDG family. However, if the identity criterion is relaxed to include residues that are identical in at least 3 of the five S1P receptors, then four out of five in TM3, nine out of ten in TM5, and five out of five are identical in TM6. Eight strictly conserved residues and an additional ten nearly conserved residues suggest that the hydrophobic binding pocket is highly conserved among these receptors. Furthermore, relative to S1P₁, S1P₅ deviates most strongly in the hydrophobic binding pocket with six differences, followed by S1P₂ and S1P₄, which each differ at four residues, and S1P₃, which only differs at three residues. Thus, the hydrophobic binding pocket shows the least diversity between S1P₁ and S1P₃. This coincides with the similar ligand properties of FTY-720-P, which at these two receptors the K_{D} is 0.21±0.17 nM and 5.0±2.7 nM, for S1P₁ and S1P₃, respectively (19). Comparison of the ligand properties with the S1P₁ specific agonist SEW2871 (22) using four mutants that showed no or greatly reduced activation by S1P indicated that three of the four residues also impaired activation to the synthetic ligand. In contrast the V6.40L mutant was slightly activated by SEW2871 but not by S1P. These results not only illustrate the general importance of the residues identified by the computationally-guided mutagenesis in S1P₁ function, but also point out that differences do exist between the individual ligands.

Significant binding occurred in several of the mutants which showed no or greatly diminished dose-dependent S1P-induced activation, indicating that these residues may play a critical role in the conformational change required for activation, but their interaction with the ligand is not essential as indicated by the retained binding. Introduction of charged residues to replace M3.32K, L3.43&3.44E, and L5.51E, severely disrupted activation and either abolished or significantly reduced (L5.51E) ligand binding compared to the WT receptor. Thus the hydrophobic environment appears to be necessary for ligand binding and consequent activation.

Some of the residues that the inventors identified as part of the hydrophobic binding pocket of S1P₁ have also been mutated in other GPCR and some were also found to play a role in ligand recognition/activation. L3.36 when mutated to alanine in the human brakykinin B2 receptor subtype did not reduce ligand affinity (37). In contrast, the inventors' L3.36G/E mutants showed altered activation properties. F5.48, when mutated to alanine in the human VIP receptor, reduced potency but not efficacy (38). F5.48G mutants both failed to show dose dependent activation by S1P but showed over 50% ligand binding relative to WT, indicating that this residue is involved in the activation of other receptors as well. There was a striking similarity between the W6.48A mutation and the melanocortin MC4R (39), cholecystokinin CCKR (40), and AA3R receptors (41), as in all instances receptor activation was reduced without loss of binding. This unique property of W6.48 is consistent with its putative role in the activation of GPCR by a diverse family of ligands. However, W6.48 does not play an identical role in the receptor most closely related to the EDG family, the cannabinoid receptor. The W6.48(357)A mutation of the CB₁ receptor displayed an enhancement of ligand-induced GTPγS binding.(42) Enhanced efficacy was also observed for some agonists at the corresponding mutant of the CCK-B/gastrin receptor.(43) Enhanced efficacy of W6.48A in concert with modeling studies and increased basal activity and lack of ligand-induced response by the CB₁ F3.36A mutant led those authors to conclude that CB₁ activation involves loss of contact between F3.36 and W6.48.(42) In contrast, the inventors' results and the refined model they have developed suggest that S1P₁ receptor activation involves formation of contact between these residues.

The inventors' model not only serves as a good template for the modeling of the other EDG receptors, but also defines the specific conformation of S1P relevant to S1P₁ agonism. This structure, in combination with the inventors' more recently published S1P₁ complex of the S1P₁-selective agonist, SEW2871,(35) define the pharmacophore for S1P₁ agonism. Superimposing the S1P₁ complex structures of S1P and SEW2871 illustrated that the phosphate group of S1P occupies the same geometric position as a trifluoromethyl group of SEW2871. Similarly, the ammonium group of S1P occupies the same space as a weakly electron-poor hydrogen atom. The remainder of each structure occupies common volume, and the superposed structures have quite similar lengths. These superposed structures define a geometric pharmacophore with distance ranges between pharmacophore elements shown in Table 5. This pharmacophore was used to identify novel lead compounds from the Enhanced NCI Database Browser. Successful identification of NCI-59474 and NCI-99548 compounds, determined by the inventors to be partial agonists of S1P₁ provides proof that in silico screening of large chemical libraries to identify novel molecular scaffolds that interact with the S1P₁ receptor is now possible.

The inventors identified F5.48G and V6.40L, exhibiting no ligand-dependent activation by S1P. Mutants L3.36E and W6.48A showed greatly reduced activation, yet all four maintained wild type [³²P]S1P binding, suggesting a role in the conformational transition of S1P₁ to its activated state. Although V6.40L was not activated by S1P, it showed partial activation by the SEW2871 ligand.

The invention will now be further described by means of the following non-limiting examples.

### EXAMPLES

### Reagents

All reagents were of analytical purity and obtained from Sigma-Aldrich (St. Louis, Mo.) unless specified otherwise. S1P was purchased from Avanti Polar Lipids (Alabaster, Ala.). SEW2871 was a generous gift from Dr. Hugh Rosen (Scripps Research Institute, San Diego).

### Residue Nomenclature

Amino acids in the transmembrane (TM) domains of S1P₁ can be assigned index positions to facilitate comparison between GPCR with different numbers of amino acids, as described by Weinstein and coworkers (29). An index position is in the format x.xx. The first number denotes the TM domain in which the residue appears. The second number indicates the position of that residue relative to the most highly conserved residue in that TM domain which is arbitrarily assigned position 50. E3.29, then, indicates the relative position of this glutamate in TM 3 relative to the highly conserved arginine 3.50 in the E(D)RY motif (29).

### Receptor Model Development: S1P₁

A model of human S1P₁ (GenBank™ accession number AFP23365) was developed by homology to a model of rhodopsin (Protein Data Bank entry 1 boj) in a manner described in the inventors' previous publications (26, 30). Briefly, the rhodopsin model was used to generate TM 1-6, while the structure for the seventh TM was based on TM7 of the dopamine D2 receptor model (31). The preliminary model was further refined by converting all cis amide bonds to the trans configuration and by manually rotating side chains at polarity-conserved positions to optimize hydrogen bonding between TM. The AMBER94 force field (32) was utilized to optimize the receptor to a 0.1 kcal/mol-A root mean square gradient. A corrected model was constructed using the preliminary model as the template with a manual realignment of TM 5 to move each residue back one position in the alignment. The corrected model was refined and minimized using the same protocol.

### Receptor Model Development: S1P, Single/Double Point Mutants

Mutant models of S1P₁ were developed by homology to the corrected S1P₁ model. Using the MOE software package, the appropriate mutation was constructed by side-chain replacement. Non-polar hydrogen atoms were added to the mutated amino acid side-chain and the model was subsequently geometry optimized. The AMBER94 force field (32) was utilized again to optimize each mutant receptor to a 0.1 kcal/mol-A root mean square gradient.

### Ligand Model Development

A computational model of sphingosine 1-phosphate (S1P) was built using the MOE software package. The phosphate group was modeled with a net-1 charge and the amine moiety was modeled with a net +1 charge. S1P was geometry optimized using the MMFF94 forcefield (33).

### Docking

Using the AUTODOCK 3.0 software package (34), S1P was docked into S1P₁ and the S1P₁ mutant receptor models. These complexes were evaluated based on final docked energy, as well as visual analysis of electrostatic and other non-bonded interactions between the ligand and receptor. Docking parameters were set to default values with the exception of the number of energy evaluations (2.5×10⁹), number of generations (30,000), local search iterations (3000) and number of runs (15). The complexes exhibiting the best interactions based on either final docked energy or visual analysis were geometry optimized using the MMFF94 force field (33) and were subjected to critical qualitative analysis. SEW2871 was docked into the S1P₁ receptor model using the same parameters and evaluation criteria.

### New Lead Identification

The docked positions of S1P and SEW2871 in the S1P₁ receptor model were superimposed and used to derive pharmacophore features sharing common locations in both structures. Distances between these common pharmacophore features comprise the pharmacophore. The pharmacophore was used to search the Enhanced NCI Database Browser (http://129.43.27.140/ncidb2/) for novel lead compounds. A trifluoromethylphenyl group was used for the anionic bioisostere, carbon atoms were used to represent the hydrophobic functionality at other pharmacophore points. Hits from the search were evaluated based on their superposition onto the S1P and SEW2871 conformations from the S1P₁ complexes. Hits were categorized as good, marginal or negative based on these superpositions. Hits were considered negative if they exceeded the volume occupied by S1P or SEW2871 due to likely steric interactions with receptor atoms.

### Site-Directed Mutagenesis

The N-terminal FLAG epitope-tagged S1P₁ receptor construct (GenBank™ accession number AF233365) was provided by Dr. Timothy Hla. Site-specific mutations were generated using the ExSite™ mutagenesis kit (Stratagene, La Jolla, Calif.) as described previously (26, 28). S1P₁ and the generated mutants were subcloned into pcDNA3.1 vector (Invitrogen, Carlsbad, Calif.). The sequence information of the mutants is listed in Table 4. Clones were verified by complete sequencing of the inserts.

TABLE 4
Description of the S1P₁ Mutant Constructs

| TM | | Sequence | | Mutated | Silent |
|---|---|---|---|---|---|
| helix | Mutation | Position | Codon | codon(s) | mutation |
| TM3 | M3.32G | 124 | ATG₃₇₂ | GGG | GGATCC |
| TM3 | M3.32K | 124 | ATG₃₇₂ | AAG | GGATCC |
| TM3 | L3.36E | 128 | CTG₃₇₈ | GAA | GCTAGC |
| TM3 | L3.36G | 128 | CTG₃₇₈ | GGA | GCTAGC |
| TM3 | V3.40L | 132 | GTG₃₉₆ | CTG | GCTAGC |
| TM3 | V3.40T | 132 | GTG₃₉₆ | ACG | GCTAGC |
| TM3 | L3.43, | 135, 136 | CTCCTC₄₀₈ | GAAGAA | CGATCG |
| | 3.44E | | | | |
| TM3 | L3.43, | 135, 136 | CTCCTC₄₀₈ | GGAGGA | CGATCG |
| | 3.44G | | | | |
| TM5 | K5.38A | 200 | AAG₆₀₀ | GCG | — |
| TM5 | I5.41A | 203 | ATC₆₀₉ | CGCGTTG | CGCGT |
| TM5 | I5.41F | 203 | ATC₆₀₉ | TTC | — |
| TM5 | F5.43G | 205 | TTC₆₁₅ | GGC | ATCGAT |
| TM5 | F5.43Y | 205 | TTC₆₁₅ | TAC | ATCGAT |
| TM5 | C5.44D | 206 | TGC₆₁₈ | GAC | ATCGAT |
| TM5 | C5.44I | 206 | TGC₆₁₈ | ATC | ATCGAT |
| TM5 | T5.45G | 207 | ACC₆₂₁ | GGT | GGTACC |
| TM5 | T5.45S | 207 | ACC₆₂₁ | AGC | CTGCAG |
| TM5 | V5.47L | 209 | GTC₆₂₇ | CTC | ATCGAT |
| TM5 | V5.47T | 209 | GTC₆₂₇ | ACC | ATCGAT |
| TM5 | F5.48Y | 210 | TTC₆₃₀ | TAC | ATCGAT |
| TM5 | F5.48G | 210 | TTC₆₃₀ | GGC | ATCGAT |
| TM5 | T5.49G | 211 | ACT₆₃₃ | GGT | — |
| TM5 | T5.49S | 211 | ACT₆₃₃ | TCT | — |
| TM5 | L5.51E | 213 | CTT₆₃₉ | GAA | ATCGAT |
| TM5 | L5.51G | 213 | CTT₆₃₉ | GGT | ATCGAT |
| TM5 | L5.52A | 214 | CTG₆₄₂ | AGCG | GCTAGC |
| TM6 | V6.37T | 258 | GTA₇₆₈ | ACA | AAGCT |
| TM6 | V6.37L | 258 | GTA₇₆₈ | CTA | AAGCT |
| TM6 | V6.40A | 261 | GTC₇₈₃ | GCA | — |
| TM6 | V6.40T | 261 | GTC₇₈₃ | ACC | GCATGC |
| TM6 | V6.40L | 261 | GTC₇₈₃ | CTC | GCATGC |
| TM6 | L6.41E | 262 | CTG₇₈₆ | GAG | GCATGC |
| TM6 | L6.41G | 262 | CTG₇₈₆ | GGG | GCATGC |
| TM6 | F6.44Y | 265 | TTC₇₉₅ | TAC | GCATGC |
| TM6 | F6.44G | 265 | TTC₇₉₅ | GGC | GCATGC |
| TM6 | W6.48E | 269 | TGG₈₀₇ | GAG | GCATGC |
| TM6 | W6.48A | 269 | TGG₈₀₇ | GCG | GCATGC |

### Cell Culture and Transfection

RH7777 and HEK-293 cells (ATCC, Manassas, Va.) were maintained in Dulbecco's modified minimal essential medium (DMEM) containing 10% fetal bovine serum (Hyclone, Logan, Utah). Cells (2×10⁶) were transfected with 2 μg of plasmid DNA with Effectene (Qiagen, Valencia, Calif.) according to the manufacturer's instructions, for 24 h. Before ligand binding and receptor activation assays, the cells were washed twice with serum-free DMEM and serum-starved for at least 6 h.

### Western Blotting

Western blot analysis of the FLAG epitope-tagged receptor construct was performed in both transiently transfected RH7777 and HEK-293 cells using a protocol described earlier (27). Anti-FLAG M2 antibody anti-β actin, and goat anti-mouse antibody conjugated with horseradish peroxidase were purchased from Sigma-Aldrich (St. Louis, Mo.) and Promega (Madison, Wis.), respectively.

### Flow Cytometry Analysis

Cell-surface expression of the FLAG-tagged S1P₁ and its mutants was determined by flow cytometry as described in the literature (27). Transfected RH7777 cells were harvested by trypsinization, and upon harvesting, the cells were maintained at 4° C. for the subsequent steps. The cells were washed with ice-cold FC buffer (phosphate buffered saline pH 7.4 (PBS) and 3% bovine serum albumin (BSA)). After washing once with FC buffer, the cells were incubated for 30 min in blocking solution (5% BSA and 5% donkey serum (Sigma) in PBS). The cells were washed once with FC buffer, and the cells were subsequently incubated for 60 min in FC buffer with the anti-FLAG M2 monoclonal antibody (Sigma) (1:200). After washing the cells twice with FC buffer, the cells were incubated for 30 min in FC buffer with the Alexa Fluor 488-labeled donkey anti-mouse IgG (Molecular Probes, Eugene, Oreg.) (1:1600). After washing the cells twice, samples were resuspended in 1% BSA in PBS and analyzed using a LSR II flow cytometer (Becton Dickinson, San Jose, Calif.). Data were analyzed with the Cell Quest software (Becton Dickinson).

### [³²P]S1P Radioligand Binding Assays

The S1P binding assays were done essentially as previously described (28). Transfected RH7777 cells (5×10⁵) were incubated at 4° C. in 20 mM Tris-HCl (pH 7.5) binding buffer containing 100 mM NaCl, 15 mM NaF, protease inhibitor cocktail (Sigma-Aldrich), and 0.2 mg/ml essentially fatty-acid free BSA with 1 nM [³²P]S1P in 50 nM S1P for 40 min. Cells were centrifuged and washed twice in binding buffer. The final pellet was resuspended in 2:1 CHCl₃/MeOH and the suspension was equilibrated in scintillation fluid overnight. Cell-bound radioactivity was measured by liquid scintillation counting using a Beckman LS5000 TA counter (Beckman Coulter, Irvine, Calif.). Specific binding was defined as the difference between total binding and non-specific binding (in the presence of 2-5 μM cold S1P). Standard errors were computed on the basis of triplicate samples from two simultaneous transfections.

For the competition assays, HEK-293 cells were used. Briefly, 4×10⁵ cells were plated in 24-well dishes and allowed to adhere overnight. The cells were then transfected with 0.4 μg of the cDNA using Lipofectamine 2000 (Invitrogen) and the transfection proceeded for 48 h. After washing the cells twice with ice-cold binding buffer (20 mM Tris-HCl, pH 7.4 and 150 mM NaCl), 0.1 nM [³²P]S1P and competing concentrations of cold S1P (1 nM-10 μM), resuspended in binding buffer+4% BSA, were applied to the cells and incubated on ice for 30 min. After washing the cells twice with ice-cold binding buffer+0.4% BSA, the cells were lysed with 0.5% SDS and equilibrated in scintillation fluid. The samples were measured in triplicate. The K_{D} and Bₘₐₓ values were Diego, Calif.).

S1P₁ Receptor Activation Assays Receptor functional assays were performed in transiently transfected RH7777 cells by measuring S1P-activated [³⁵S]GTPγS binding as previously described (28).

### Statistical Analysis

The significance of differences was determined by one-way ANOVA, Bonferroni post-hoc test using Prism statistical software (GraphPad, San Diego, Calif.). Values were considered significantly different at p<0.05.

### Results

### Mutagenesis Strategy

The previously reported computationally modeled complex of S1P in S1P₁ features 15 amino acid residues in TM 3, 4 and 6 with atoms within 4.5 Å of S1P. In the present study the inventors pursued a three-pronged replacement strategy of these residues: First, property-conserving mutations of these residues were introduced that either reduced or increased size in order to probe the impact of increased or relaxed steric constraints in the hydrophobic binding pocket on ligand-induced activation. Additionally, many of these residues were replaced with charged amino acids of similar size to probe whether disruption to hydrophobicity in the putative binding pocket would have an impact on receptor function. Third, in a few cases charged residues were replaced with non-charged residues of similar size to test the effect of polar interactions between the ligand and the receptor.

### Theoretical Model of the S1P₁—S1P Complex—Revisions of the Previous Model

Discrepancies between model-derived predictions and experimental observations in this and a previous study on S1P₄ (27) involved residues localized at the extracellular end of TM5. The differences the inventors found were not consistent with proposed structural differences between active and inactive GPCR conformations, and thus appear to be an error in the previous S1P₁ model (26,28). A corrected model of S1P₁ was built based on an alternative alignment of TM5 derived from the recently validated S1P₄ model (27). The corrected model demonstrates that 8 residues in TM5 have atoms within 4.5 Å of S1P. One of these residues, K5.38, forms an ion pair with the phosphate group of S1P. This polar interaction was not identified in previous validation of the S1P₁ model due to the incorrect positioning of amino acid residues at the top of TM5.

### Cell Surface Expression of S1P₁ Mutants

In order to verify that the WT and mutant constructs were expressed at comparable levels, membrane fractions were prepared and analyzed for expression by Western blot analysis using the N-terminal FLAG epitope present in the constructs. The levels of expression on the membrane fractions were comparable to that of the WT receptor. FC analysis was used to determine if cell surface expression of the N-terminal FLAG epitope was similar for the mutant constructs to that of the WT (Table 5, which lists results for expression of pcDNA3.1 vector-transfected control, wild type S1P₁, and mutants which displayed no or diminished S1P-induced activation, in RH7777 cells examined by flow cytometry. Expression was detected with anti FLAG M2 monoclonal antibody. Flow cytometry was performed as described in “Experimental Procedures”. Four independent experiments were conducted.). The WT and the mutant constructs, with the exception of L6.41G, were expressed at the cell surface in similar levels based on immuno-labelling for the FLAG epitope. Because the L6.41 G mutant protein was expressed in the cell lysate at a level similar to the other mutants but we were unable to detect it at the cell surface in multiple experiments, the inventors concluded that this mutation adversely affected the targeting of the receptor to the cell surface. The L6.41 E mutant was expressed and included in the pharmacological testing.

TABLE 5
Cell Surface Expression of Vector, WT S1P₁ and Mutants
With No S1P-induced Activation in RH7777 Cells

| | % Expression of |
|---|---|
| | construct from |
| Construct | total population |
| vector | 4.3 |
| WT | 9.5 |
| M3.32K | 19.3 |
| L3.36E | 15.2 |
| L3.43, 3.44E | 10.3 |
| F5.48G | 22.1 |
| L5.51E | 17.4 |
| L5.52A | 14.1 |
| V6.40L | 19.6 |
| L6.41G | 5.6 |
| W6.48A | 12.6 |

### The Effects of Mutations of Residues Lining Predicted Hydrophobic Binding Pocket on Ligand-induced Activation of S1P₁

In the first round of pharmacological testing the inventors evaluated the impact of all amino acid replacements on the EC₅₀ and maximal activation (Eₘₐₓ) elicited by S1P. The summary of the pharmacological properties caused by these replacements is presented in Table 3 A-E. After the first round of GTPγS activation experiments were completed, it became apparent that of 15 residues mutated on the basis of the previously published S1P₁ model, 13 produced changes in receptor activation with the exception of two residues in TM5, F5.43 and T5.49. In addition, parallel studies carried out on S1P₄ (27) revealed that the inventors' model needed revisions with regard to the orientation of the top half of TM5. The position of F5.43 was shifted by one position in the helix compared to the old model, causing a 1000 difference in its orientation. Mutations of F5.43 and T5.49 had the least impact on S1P activation (Table 6). Replacement of T5.49 with G or S, which removed the polar side chain and reduced the residue size, respectively, had no effect of Eₘₐₓ and increased EC₅₀ by three-fold. The Y and G replacement of F5.43, introducing a polar residue or one with decreased electron density and size, respectively, caused a modest reduction in Eₘₐₓ and an approximately ten-fold increase in EC₅₀. These residues, therefore, point away from the ligand, consistent with the modest impact (Table 6) replacements to these residues caused and the role they play in the hydrophobic binding pocket.

TABLE 6
The EC₅₀ and Eₘₐₓ Values for the Mutants
Which Retained S1P-induced Activation

| | % WT | | Nature of Amino Acid |
|---|---|---|---|
| Constructs | Eₘₐₓ + s.d. | EC₅₀ + s.d. | (AA) replacement |
| WT-S1P₁ | 100 + 9.13 | 1.4 ± 0.7 | |
| Vector | ∞ | ∞ | |
| F5.43Y | 90 + 7 | 23 + 4.7 | Introduction of polar |
| | | | functional group |
| F5.43G | 70 + 6 | 11.8 + 3.9 | Decrease in electron |
| | | | density and size |
| T5.49G | 107 + 1.4 | 5.4 + 0.56 | Removal of polar side |
| | | | chain |
| T5.49S | 168 + 15 | 6.6 + 1.2 | Reduction in size |

The refined model was used to identify an additional 5 residues from TM5 within 4.5 Å of S1P for a second round of pharmacological testing. Out the 20 residues reported here, S1P-induced activation was altered for 18 residues.

The inventors found that replacements of seven residues depicted in Table 7 caused either marked decreases in Eₘₐₓ or increased EC₅₀ substantially. These findings corroborate the predictions of the model and appear to be consistent with the hypothesis that even conservative mutational replacements of these amino acids in close proximity of the aliphatic part of S1P have a marked impact on the function of the receptor.

TABLE 7
The EC₅₀ and Eₘₐₓ Values for the Mutants Which Displayed Partial
Activation or Increased EC₅₀ Compared
to the Wild Type (WT)

| | % WT | | Nature of Amino Acid |
|---|---|---|---|
| Constructs | Eₘₐₓ + s.d. | EC₅₀ + s.d. | (AA) replacement |
| WT-S1P₁ | 100 + 9.13 | 1.4 ± 0.7 | |
| Vector | ∞ | ∞ | |
| L3.36E | 39 ± 2.8 | 0.62 ± 0.46 | Change from a nonpolar |
| | | | to a charged AA |
| L3.36G | 75 + 12 | 23 ± 8 | Reduction in size |
| V3.40T | 137 + 10 | 13.8 ± 1.8 | Change from a nonpolar |
| | | | to a charged AA |
| V3.40L | 55 + 6.3 | 7.0 ± 4.5 | Increase in size |
| K5.38A | 97 + 15 | 32.4 + 8 | Decrease in electron |
| | | | density and size of AA |
| I5.41A | 26 + 4 | 157.2 + 35 | Reduction in size |
| I5.41F | 27 + 3.4 | 18.9 + 5 | Increase in size and |
| | | | electron density |
| C5.44D | 85 + 13 | 79 ± 17 | Increase in polarity |
| C5.44I | 37 + 2.3 | 20.4 ± 2.1 | Removal of polar side |
| | | | chain |
| T5.45G | 96 + 18 | 30.9 + 8.5 | Removal of polar side |
| | | | chain |
| T5.45S | 28 + 4 | 7.9 + 3 | Reduction in size |
| L5.51E | ∞ | ∞ | Change from a nonpolar |
| | | | to a charged AA |
| L5.51G | 46 + 8 | 2.6 ± 2.1 | Reduction in size |
| V6.37T | 181 + 26 | 13 + 2.4 | Change from a nonpolar |
| | | | to a polar AA |
| V6.37L | 247 + 11.3 | 37.6 + 8.4 | Increase in size of AA |
| F6.44Y | 37 + 2.6 | 3.7 ± 3.3 | Introduction of polar |
| | | | functional AA |
| F6.44G | ∞ | ∞ | Decrease in electron |
| | | | density and size of AA |
| W6.48A | 34 ± 1.5 | 13 ± 4.2 | Decrease in electron |
| | | | density and size of AA |

Mutants at four residues showed significant variability in S1P-induced response depending on the type of substitution. However, for these residues one of the replacement mutants remained responsive to ligand, sometimes with little or more pronounced decrease in activation (Table 8). Substitution of a charged moiety for M3.32 led to a receptor that showed no dose-dependent activation by S1P. Reduction in size of L6.41 and increase in size of V5.47 and V6.40 led to the loss of ligand-induced activation. In contrast, decreases in size at M3.32 and V6.40, changes polarity at V5.47 and V6.40 or addition of charge at L6.41 resulted in less drastic effects on ligand-induced receptor activation.

TABLE 8
The EC₅₀ and Eₘₐₓ Values for the Residues Displaying
Variability Depending on the Amino Acid Substituted

| | % WT | | Nature of Amino Acid |
|---|---|---|---|
| Constructs | Eₘₐₓ + s.d. | EC₅₀ + s.d. | (AA) replacement |
| WT-S1P₁ | 100 + 9.13 | 1.4 ± 0.7 | |
| Vector | ∞ | ∞ | |
| M3.32K | ∞ | ∞ | Change from a nonpolar |
| | | | to a charged AA |
| M3.32G | 77 + 5 | 35 ± 5 | Reduction in size |
| V5.47T | 100 + 3 | 89 + 15 | Change from a nonpolar |
| | | | to a polar AA |
| V5.47L | ∞ | ∞ | Increase in size |
| V6.40A | 69 + 4.4 | 14.8 + 3.8 | Reduction in size |
| V6.40T | 61 + 9.4 | 57.3 + 25 | Change from a nonpolar |
| | | | to a polar AA |
| V6.40L | ∞ | ∞ | Increase in size |
| L6.41E | 59 + 1.8 | 4.1 ± 0.63 | Change from a nonpolar |
| | | | to a charged AA |
| L6.41G | ∞ | ∞ | Reduction in size |

The computational model placed leucine L3.43 at the bottom of the hydrophobic binding pocket. Positions 3.43 and 3.44 are a conserved LL motif in all S1P receptors, therefore their combined importance was tested by simultaneously replacing both residues with either a charged glutamic acid or smaller glycine. Either replacement caused a complete loss of ligand-induced activation (Table 9). These mutants, similar to those that were non-functional (listed in Table 8) always showed less than 20% of the basal GTPγS binding, indicating that they were not constitutively active.

TABLE 9
The EC₅₀ and Eₘₐₓ Values for the Double Mutants

| | % WT | | Nature of Amino Acid |
|---|---|---|---|
| Constructs | Eₘₐₓ + s.d. | EC₅₀ + s.d. | (AA) replacement |
| WT-S1P₁ | 100 + 9.13 | 1.4 ± 0.7 | |
| Vector | ∞ | ∞ | |
| L3.43, 3.44E | ∞ | ∞ | Change from a nonpolar |
| | | | to a charged AA |
| L3.43, 3.44G | ∞ | ∞ | Reduction in size |

The inventors noted that in TM 5 and 6 there were three residues, F5.48, L5.52, and W6.48, when altered either in charge or in size either completely or substantially lost their ligand-induced activation to S1P (Table 10). To further characterize the ligand-induced activation of these mutants, the inventors also exposed them to SEW2871, a recently-identified non-lipid agonist of S1P receptors (35). L3.36E, F5.48G, and W6.48A showed similarly impaired activation to SEW2871 to that seen with S1P. Unexpectedly, V6.40L showed a dose-dependent partial activation with SE2871.

TABLE 10*
The EC₅₀ and Eₘₐₓ Values for Mutations
in Which Activation Was Abolished

| | % WT | | Nature of Amino Acid |
|---|---|---|---|
| Constructs | Eₘₐₓ + s.d. | EC₅₀ + s.d. | (AA) replacement |
| WT-S1P₁ | 100 ± 9.13 | 1.4 + 0.7 | |
| Vector | ∞ | ∞ | |
| F5.48Y | ∞ | ∞ | Increase in polarity |
| F5.48G | ∞ | ∞ | Decrease in electron |
| | | | density and size |
| L5.52A | ∞ | ∞ | Reduction in size |
| W6.48E | ∞ | ∞ | Decrease in electron |
| | | | density, size, and |
| | | | introduction of a |
| | | | charged AA |

*S1P-induced receptor activation. Ligand-induced [³⁵S]GTPγS binding was calculated as the difference between binding to 5-10 μg of membrane fraction in the presence and absence of ligand. EC₅₀ values for S1P-induced (0.1 nM-10 μM) GTPγS activation was measured in RH7777 cells transfected with empty vector, WT S1P₁, and each mutant. Eₘₐₓ values were calculated as the maximal responses normalized to the WT maximal response. Two independent transfections were performed. Data points represent the mean + SD (n = 3).

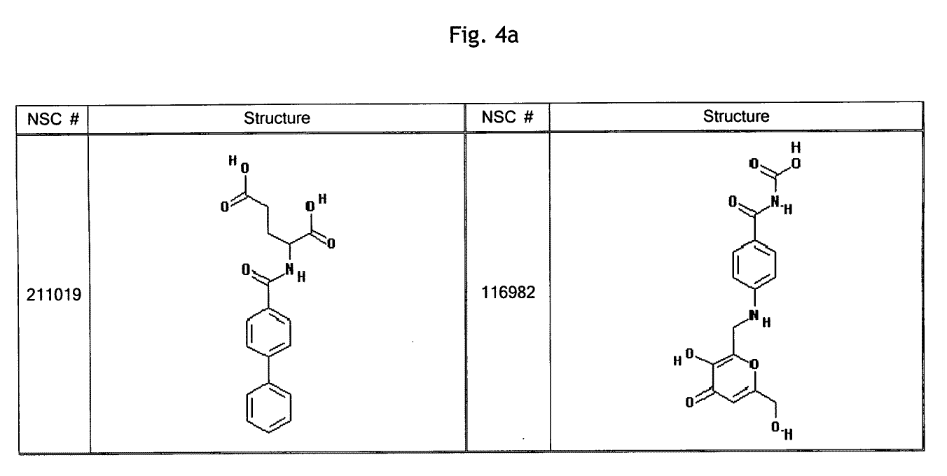

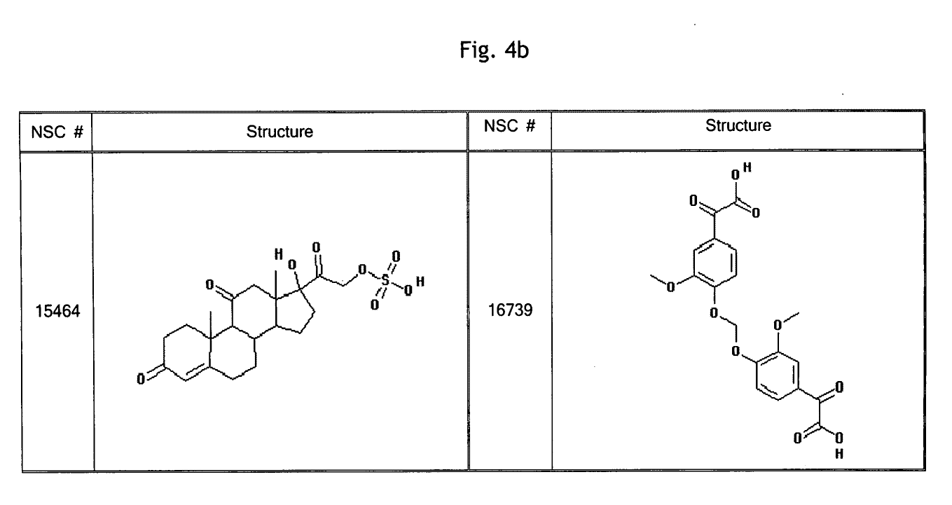

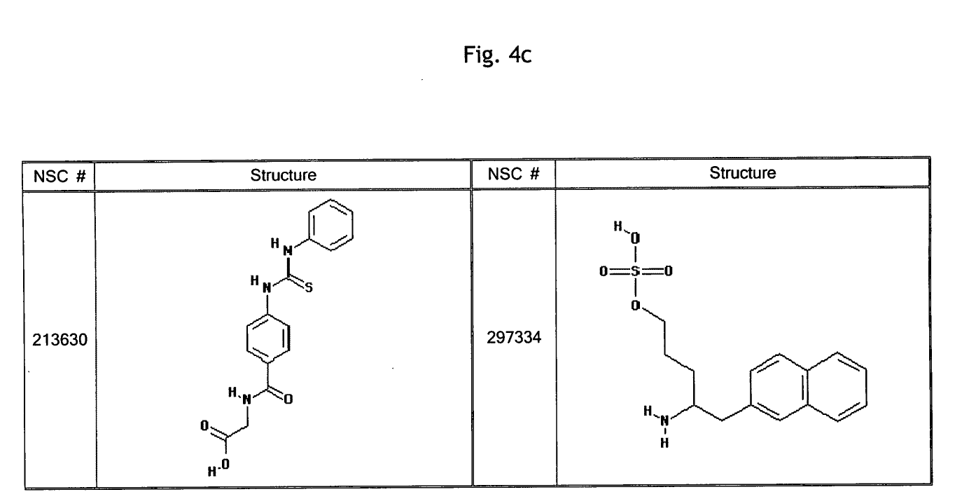

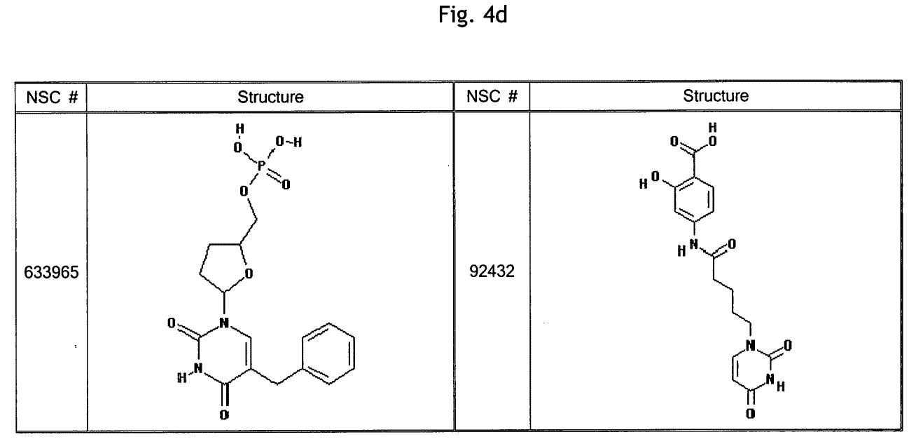

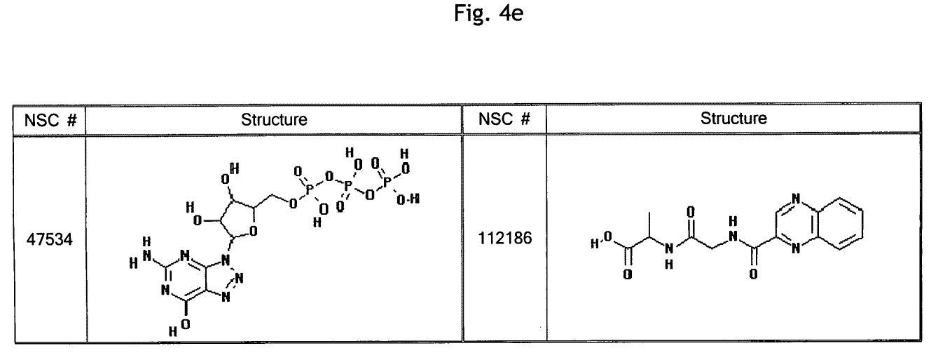

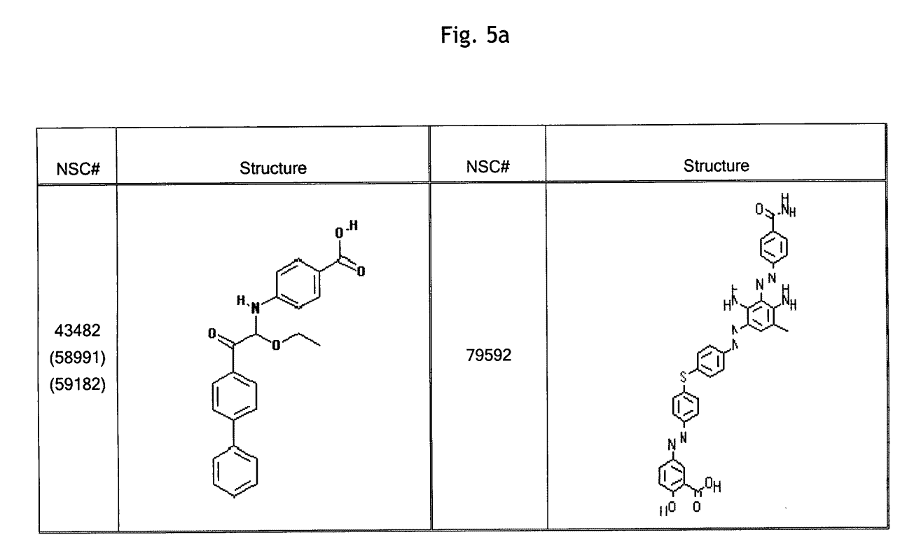

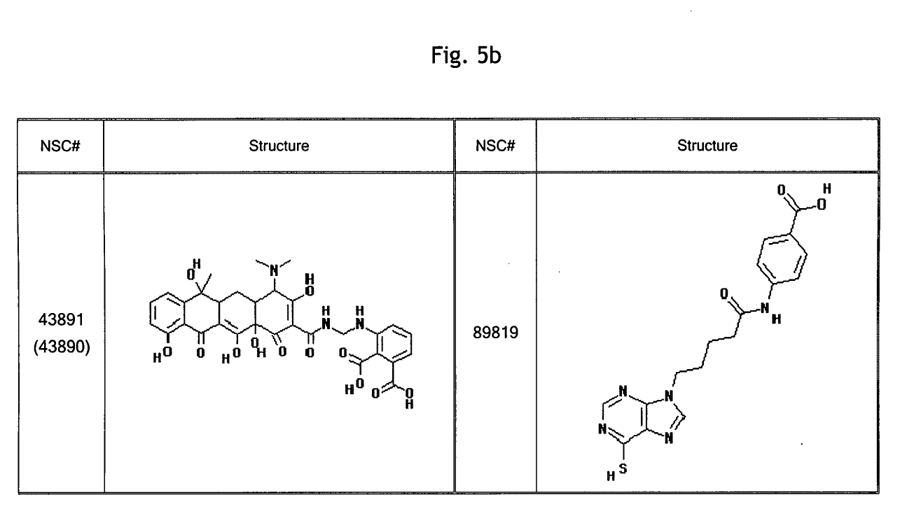

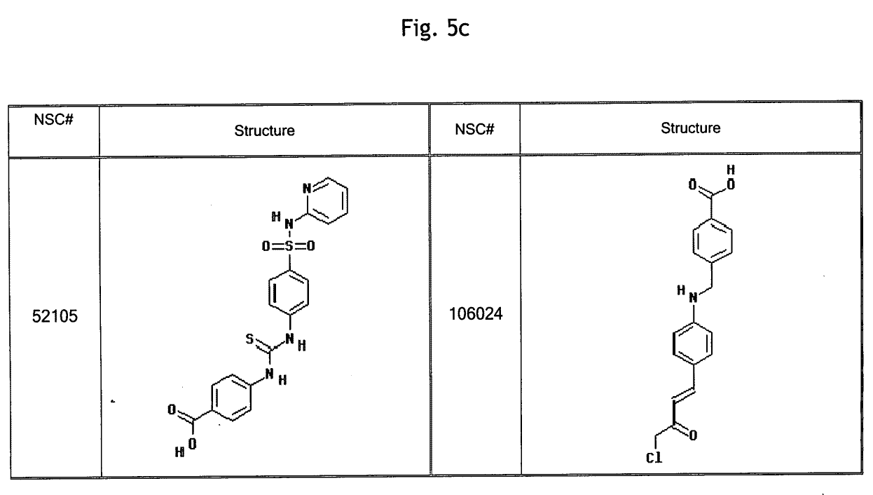

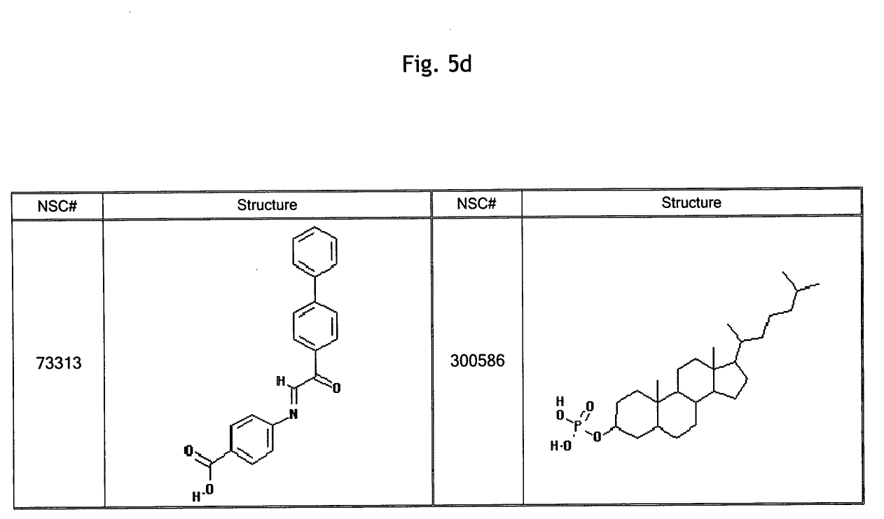

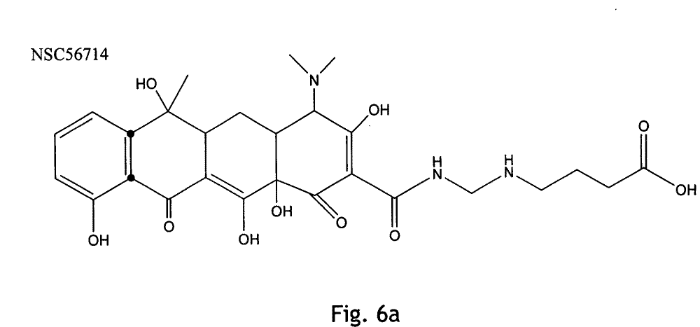

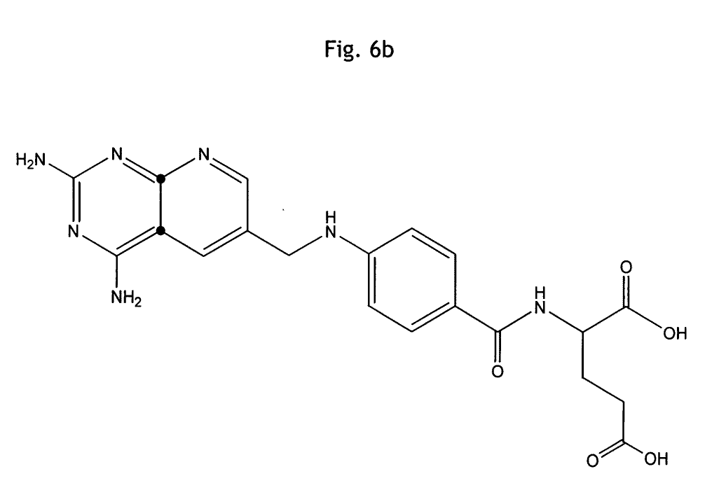

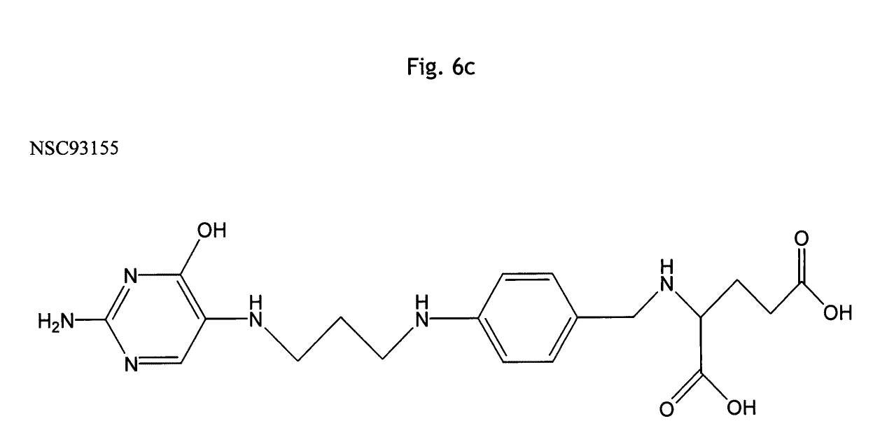

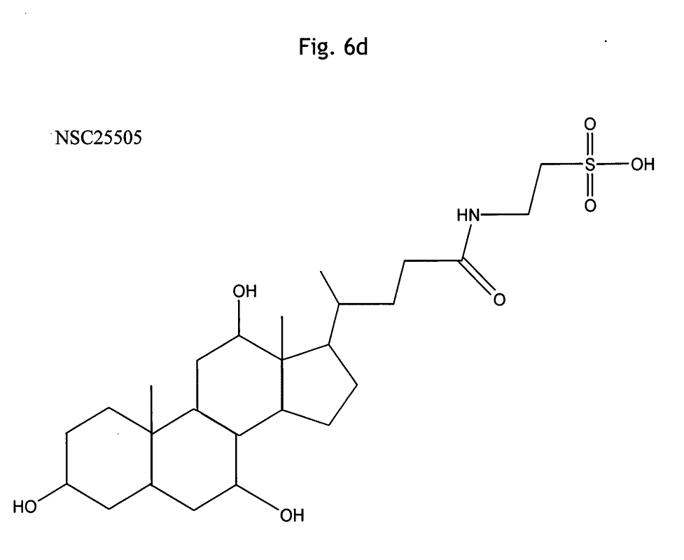

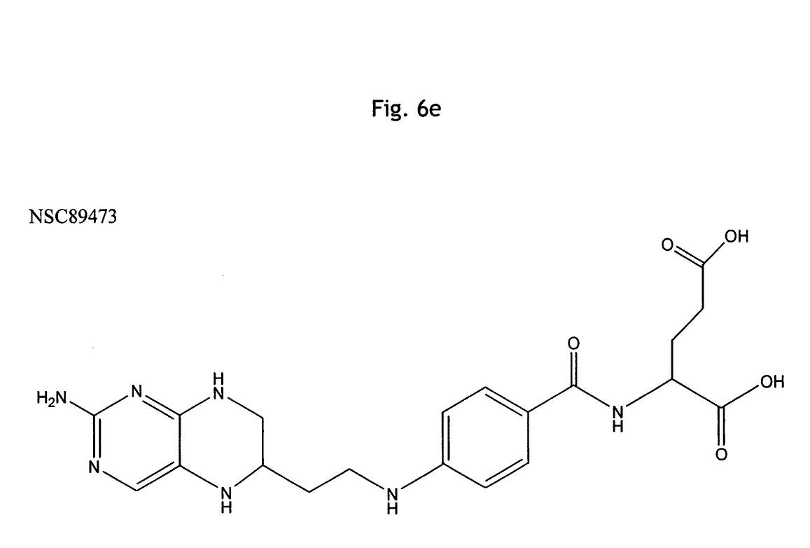

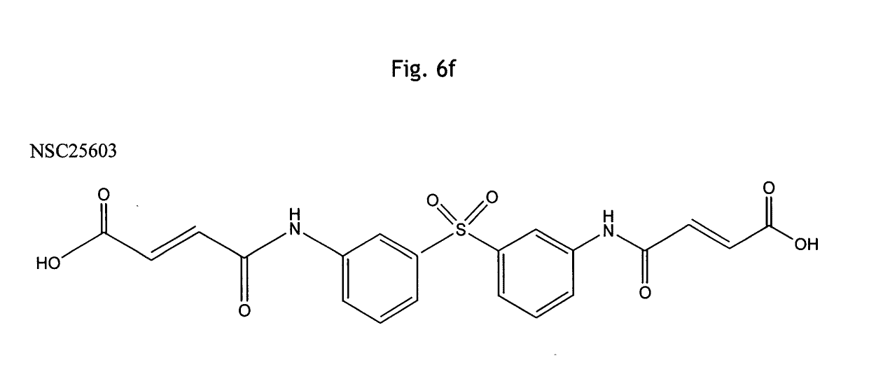

### [³²P]S1P Binding to S1P₁ Mutants Without Ligand-Induced Activation

[³²P]S1P radioligand binding studies were performed with mutants M3.32K, L3.36E, L3.43, 3.44E, L3.43, 3, 0.44G, C5.44D, V5.47L, V5.47T, F5.48G, L5.51E, L5.52A, V6.40L, L6.41G, and W6.48A, demonstrating much impaired dose-dependent activation by S1P in the GTPγS activation experiments. In the inventors' system the apparent K_{D} for S1P binding at the WT receptor was 36±2 nM (28). Therefore, a radioligand concentration of 50 nM was chosen to test whether those mutants that lacked activation would maintain some degree of S1P binding. Compared to the vector transfected cells, nine mutants out of the thirteen tested showed significant ligand binding (FIG. 4). Out of these nine with significant ligand binding, four mutants, L3.36E, F5.48G, V6.40L and W6.48A showed binding in excess of 50% of the WT. Thus, these replacements introduced in the putative hydrophobic ligand binding pocket had a pronounced tendency to affect ligand activation while maintaining some degree of ligand binding, suggesting that these residues play a crucial role in receptor activation, rather than solely ligand binding. The model demonstrates that L3.36 and W6.48 make good van der Waals contact with each other. The detrimental effect of mutation at either of these sites on receptor activation, but not ligand binding, suggests that this van der Waals contact is unique to the activated conformation of S1P₁. Docked complexes of S1P with these mutants displayed the polar interactions typical of the wild-type complex. This finding is consistent with the observed S1P binding by these mutants. The complexes additionally indicate that the hydrophobic tail of S1P does not extend as deeply into the hydrophobic pocket. The hydrophobic tail of S1P instead occupies the empty volume produced upon mutation of L3.36 or W6.48 to smaller or less branched residues. This difference in position for the hydrophobic tail of S1P is consistent with the lack of activation observed for these mutants.

The competition binding showed that the mutants, L3.36E, F5.48G, V6.40A, and W6.48A, which displayed at least 50% of the specific binding of the WT, had K_{D} and Bₘₐₓ comparable to the WT S1P₁. These data further validate the importance of the role which these residues play in the functionality, but not the binding of S1P to the S1P₁ receptor.

### Identification of New S1P, Agonist Scaffolds

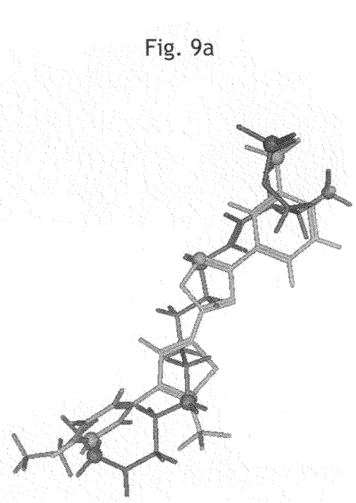

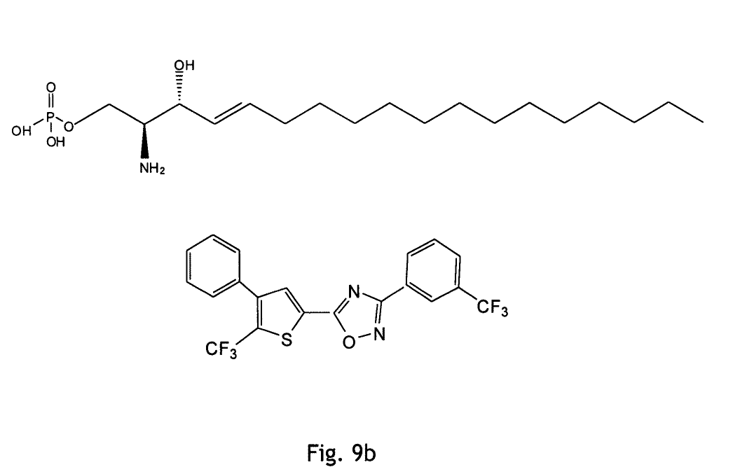

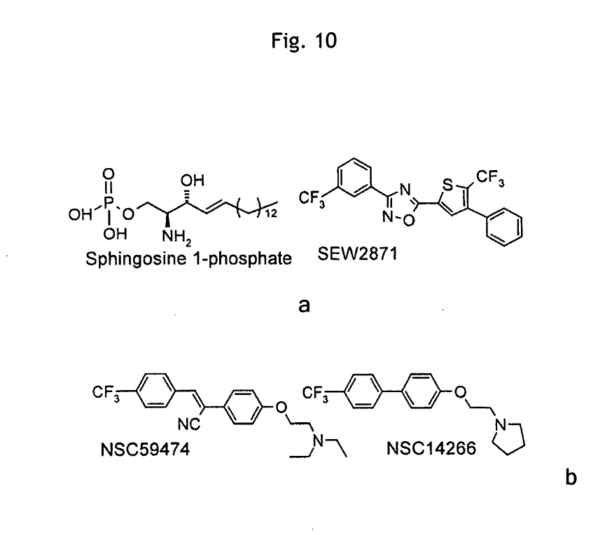

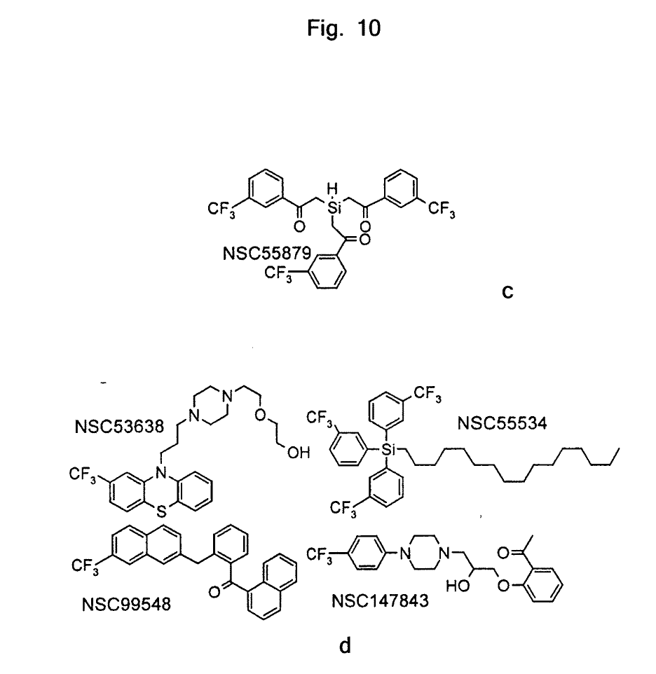

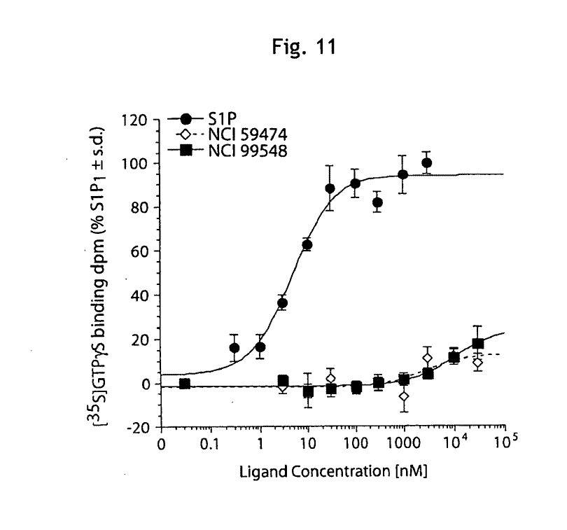

Docked complexes of two structurally distinct S1P₁ receptor agonists, S1P and SEW2871, were used to derive a pharmacopore describing the important chemical functional moieties and distances between those moieties that produce S1P₁ receptor activation. The superposed structures of S1P and SEW2871 from the docked complexes are shown in FIG. 9 and the distance ranges between pharmacophore elements are shown in Table 12. A subset of these distances was used to identify 13 hits in the Enhanced NCI Database Browser using a trifluoromethylphenyl group to provide the anionic bioisostere. Three hits were eliminated due to poor superposition on the known agonists. The remaining ten hits were categorized based on the quality of their rigid superposition onto the known agonist structures. Four hits were considered good matches (NSC146266, 145964, 59474, and 75030). Two hits were considered marginal matches (NSC55879 and 68644). Four hits were considered negatives, with additional bulk or incorrect curvature (NSC147843, 53638, 55534 and 99548). The ten hits were requested from the NCI Developmental Therapeutics Program.

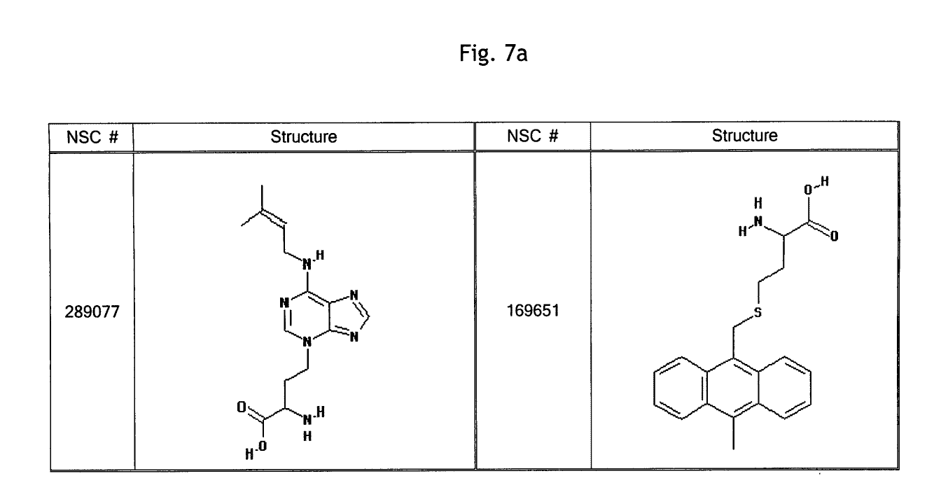

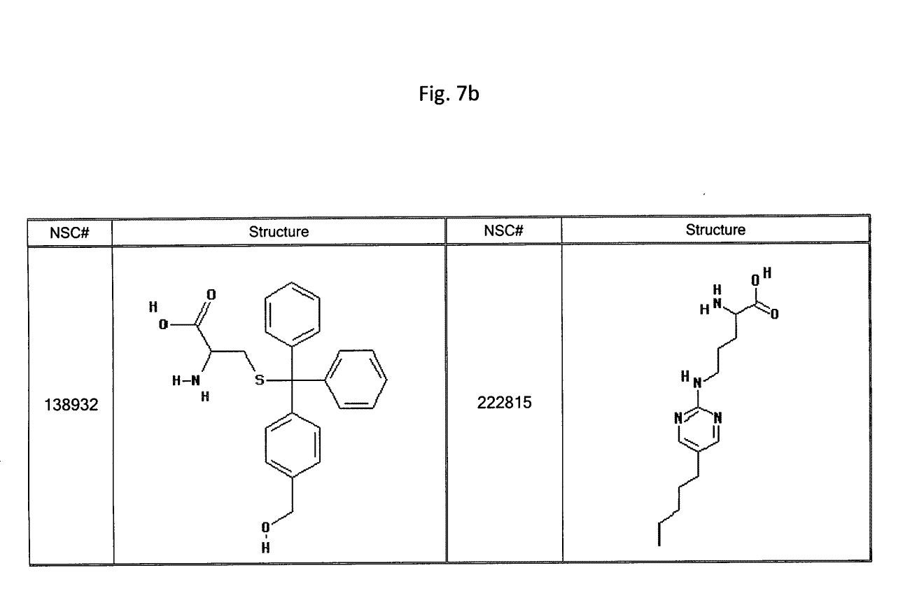

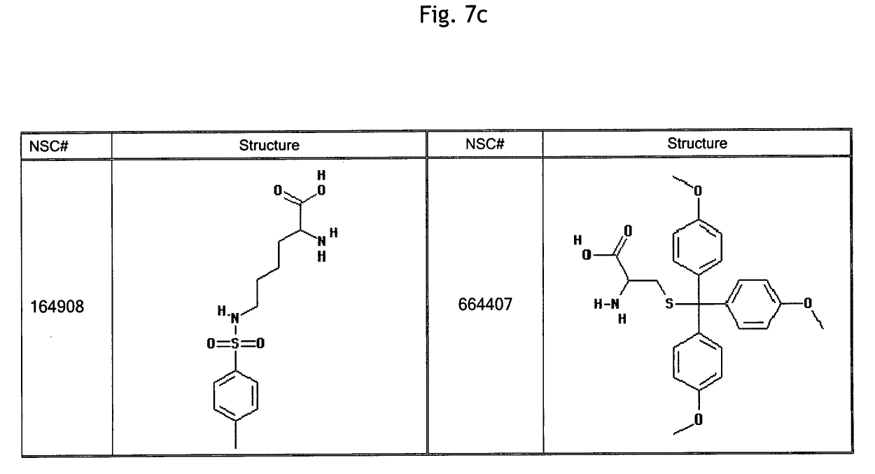

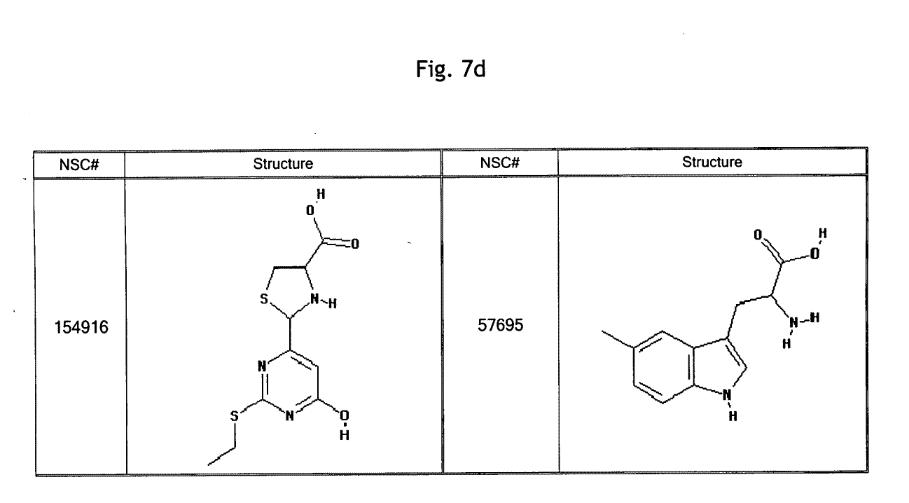

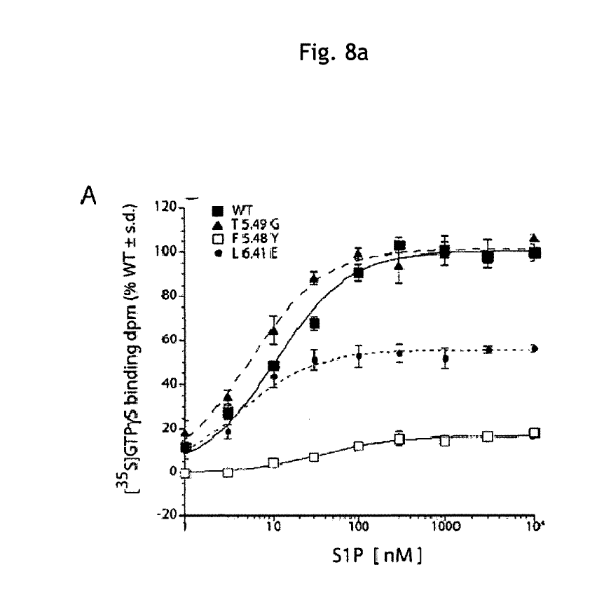

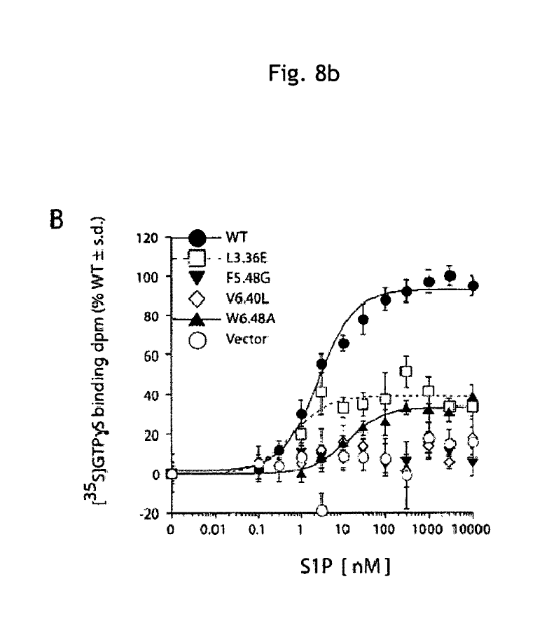

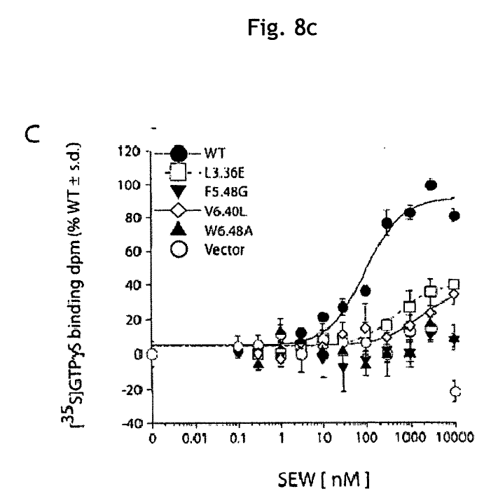

Samples of seven compounds were available (Good —NSC146266 and 59474; Marginal—55879; Negative—147843, 53638, 55534 and 99548, FIG. 7A) and were screened for S1P₁ receptor activation in the GTPγS activation assay. Two of the seven showed GTPγS activation greater than that of the vehicle at 10 μM, NSC59474 and 99548. Dose response curves were then generated.

TABLE 11
K_{D} and Bₘₐₓ Values of the Mutants Which Displayed at Least
50% Specific Binding of the WT, S1P₁.

| Construct | K_{D} + s.d. [nM] | Bₘₐₓ (fmol) |
|---|---|---|
| WT-S1P₁ | 38.7 + 1.2 | 2450 |
| L3.36E | 27.8 + 1.15 | 1980 |
| F5.48G | 20.2 + 1.2 | 2580 |
| V6.40A | 27.6 + 1.1 | 2040 |
| W6.48A | 38.5 + 1.15 | 2080 |

TABLE 12
Average S1P₁ Agonist Pharmacophore Distances (Å)
Based on Complexes With S1P and SEW2871

| | a | b | c | d | e |
|---|---|---|---|---|---|
| a | | 3.2 | 6.0 | 11.4 | 14.8 |
| b | | | 6.2 | 10.6 | 14.7 |
| c | | | | 6.0 | 9.2 |
| d | | | | | 5.0 |

### REFERENCES

1. Hla, T. (2003) Pharmacol Res 47(5), 401-407
2. Spiegel, S., and Milstien, S. (2003) Nat Rev Mol Cell Biol 4(5), 397-407
3. Hla, T. (2004) Semin Cell Dev Biol 15(5), 513-520
4. Liu, Y., Wada, R., Yamashita, T., Mi, Y., Deng, C.-X., Hobson, J. P., Rosenfeldt, H. M., Nava, V. E., Chae, S.-S., Lee, M.-J., Liu, C. H., Hla, T., Spiegel, S., and Proia, R. L. (2000) J. Clin. Invest. 106(8), 951-961
5. Kupperman, E., An, S., Osborne, N., Waldron, S., and Stainier, D. Y. R. (2000) Nature 406, 192-195
6. Brinkmann, V., Cyster, J. G., and Hla, T. (2004) Am J Transplant 4(7), 1019-1025
7. Chae, S. S., Paik, J. H., Furneaux, H., and Hla, T. (2004) J Clin Invest 114(8), 1082-1089
8. Tigyi, G., and Parrill, A. L. (2003) Prog Lipid Res 42(6), 498-526
9. Parrill, A. L., Sardar, V. M., and Yuan, H. (2004) Semin Cell Dev Biol 15(5), 467-476
10. Taha, T. A., Argraves, K. M., and Obeid, L. M. (2004) Biochim Biophys Acta 1682(1-3), 48-55
11. Ghosh, T. K., Bian, J., and Gill, D. L. (1990) Science 248(4963), 1653-1656
12. Ghosh, T. K., Bian, J., and Gill, D. L. (1994) J Biol Chem 269(36), 22628-22635
13. Meyer zu Heringdorf, D., Liliom, K., Schaefer, M., Danneberg, K., Jaggar, J. H., Tigyi, G., and Jakobs, K. H. (2003) FEBS Lett 554(3), 443-449
14. Itagaki, K., and Hauser, C. J. (2003) J Biol Chem 278(30), 27540-27547
15. Olivera, A., Rosenfeldt, H. M., Bektas, M., Wang, F., Ishii, I., Chun, J., Milstien, S., and Spiegel, S. (2003) J Biol Chem 278(47), 46452-46460
16. Chun, J., Goetzl, E. J., Hla, T., Igarashi, Y., Lynch, K. R., Moolenaar, W., Pyne, S., and Tigyi, G. (2002) Pharmacol Rev 54(2), 265-269
17. Brinkmann, V., Pinschewer, D. D., Feng, L., and Chen, S. (2001) Transplantation 72(5), 764-769
18. Brinkmann, V., and Lynch, K. R. (2002) Curr. Opin. Immunol. 14, 569-575
19. Mandala, S., Hajdu, R., Bergstrom, J., Quackenbush, E., Xie, J., Milligan, J., Thornton, R., Shei, G., Card, D., Keohane, C., Rosenbach, M., Hale, J., Lynch, C. L., Rupprecht, K., Parsons, W., and Rosen, H. (2002) Science 296, 346-349
20. Buenemann, M., Brandts, B. K., Pott, L., Liliom, K., Tseng, J.-L., Desiderio, D. M., Sun, G., Miller, D., and Tigyi, G. (1996) EMBO J. 15, 5524-5537
21. Liliom, K., Sun, G., Bunemann, M., Virag, T., Nusser, N., Baker, D. L., Wang, D. A., Fabian, M. J., Brandts, B., Bender, K., Eickel, A., Malik, K. U., Miller, D. D., Desiderio, D. M., Tigyi, G., and Pott, L. (2001) Biochem J 355(Pt 1), 189-197
22. Sanna, M. G., Liao, J., Jo, E., Alfonso, C., Ahn, M. Y., Peterson, M. S., Webb, B., Lefebvre, S., Chun, J., Gray, N., and Rosen, H. (2004) J Biol Chem 279(14), 13839-13848
23. Cinamon, G., Matloubian, M., Lesneski, M. J., Xu, Y., Low, C., Lu, T., Proia, R. L., and Cyster, J. G. (2004) Nat Immunol 5(7), 713-720
24. Graeler, M., and Goetzl, E. J. (2002) Faseb J 16(14), 1874-1878
25. Graeler, M. H., Kong, Y., Karliner, J. S., and Goetzl, E. J. (2003) J Biol Chem 278(30), 27737-27741
26. Parrill, A. L., Wang, D., Bautista, D. L., Van Brocklyn, J. R., Lorincz, Z., Fischer, D. J., Baker, D. L., Liliom, K., Spiegel, S., and Tigyi, G. (2000) J Biol Chem 275(50), 39379-39384
27. Inagaki, Y., Pham, T. T., Fujiwara, Y., Kohno, T., Osborne, D. A., Igarashi, Y., Tigyi, G., and Parrill, A. L. (2005) Biochem J 389(Pt 1), 187-195
28. Wang, D. A., Lorincz, Z., Bautista, D. L., Liliom, K., Tigyi, G., and Parrill, A. L. (2001) J Biol Chem 276(52), 49213-49220
29. Ballesteros, J. A., and Weinstein, H. (1995) Chapter 19. In: Conn, P. M., and Sealfon, S. C. (eds). Methods in Neurosciences, Academic Press, San Diego
30. Bautista, D. L., Baker, D. L., Wang, D., Fischer, D. J., Van Brocklyn, J., Spiegel, S., Tigyi, G., and Parrill, A. L. (2000) J. Mol. Struct. THEOCHEM 529(1-3), 219-224
31. Konvicka, K., Ballesteros, J. A., and Weinstein, H. (1998) Biophys. J. 75, 601-611
32. Cornell, W. D., Cieplak, P., Bayl), C. I., Gould, I. R., Merz, K. M. J., Ferguson, D. M., Spellmeyer, D. C., Fox, T., Caldwell, J. W., and Kollman, P. A. (1995) J. Am. Chem. Soc. 117(19), 5179-5197
33. Halgren, T. A. (1996) J. Comp. Chem. 17(5 & 6), 490-519
34. Morris, G. M., Goodsell, D. S., Halliday, R. S., Huey, R., Hart, W. E., Belew, R. K., and Olson, A. J. (1998) J. Comput. Chem. 19, 1639-1662
35. Jo, E., Sanna, M. G., Gonzalez-Cabrera, P. J., Thangada, S., Tigyi, G., Osborne, D. A., Hla, T., Parrill, A. L., and Rosen, H. (2005) Chem Biol 12(6), 703-715
36. Yamamura, S., Yatomi, Y., Ruan, F., Sweeney, E. A., Hakomori, S., and Igarashi, Y. (1997) Biochemistry 36(35), 10751-10759
37. Meini, S., Cucchi, P., Bellucci, F., Catalani, C., Faiella, A., Rotondaro, L., Quartara, L., Giolitti, A., and Maggi, C. A. (2004) Biochem Pharmacol 67(4), 601-609
38. Di Paolo, E., Vilardaga, J. P., Petry, H., Moguilevsky, N., Bollen, A., Robberecht, P., and Waelbroeck, M. (1999) Peptides 20(10), 1187-1193
39. Yang, Y. K., Fong, T. M., Dickinson, C. J., Mao, C., Li, J. Y., Tota, M. R., Mosley, R., Van Der Ploeg, L. H., and Gantz, I. (2000) Biochemistry 39(48), 14900-14911
40. Escrieut, C., Gigoux, V., Archer, E., Verrier, S., Maigret, B., Behrendt, R., Moroder, L., Bignon, E., Silvente-Poirot, S., Pradayrol, L., and Fourmy, D. (2002) J Biol Chem 277(9), 7546-7555
41. Gao, Z. G., Chen, A., Barak, D., Kim, S. K., Muller, C. E., and Jacobson, K. A. (2002) J Biol Chem 277(21), 19056-19063
42. McAllister, S. D., Hurst, D. P., Barnett-Norris, J., Lynch, D., Reggio, P. H., and Abood, M. E. (2004) J Biol Chem 279(46), 48024-48037
43. Blaker, M., Ren, Y., Seshadri, L., McBride, E. W., Beinborn, M., and Kopin, A. S. (2000) Mol Pharmacol 58(2), 399-406

What is claimed is:

1. A method for identifying and distinguishing compounds having LPA receptor agonist, LPA receptor antagonist, S1P agonist, or S1P antagonist activity, the method comprising
providing pharmacophore features and distances between features as described by an LPA receptor ligand pharmacophore, an S1P receptor ligand pharmacophore, or both, as input to a 3-dimensional database;
screening resultant matches by rigidly docking conformation matched to the pharmacophore into the receptor model; and
selecting structures for experimental screening based on their size and electronic complementarity to the receptor model.

2. A method as in claim 1 wherein the LPA receptor ligand pharmacophore comprises pharmacophore features of Scheme 1 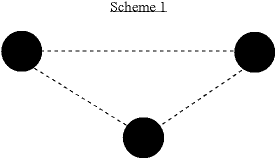
where:
A is an anionic functional group;
B and C are hydrophobic functional groups;
an LPA₁ Antagonist (A) has a distance between A and B of 7-11 Å, a distance between B and C of 6-10 Å, and a distance between A and C of 8-12 Å;
an LPA₁ Antagonist (B) has a distance between A and B of 7-11 Å, a distance between B and C of 5-8 Å, and a distance between A and C of 6-12 Å;
an LPA₁ Agonist has a distance between A and B of 15-17 Å, a distance between B and C of 9.2-11.2 Å, a distance between A and C of 15.5-17.5 Å;
an LPA₂ Antagonist has a distance between A and B of 5-9 Å, a distance between B and C of 4-7 Å, and a distance between A and C of 4-6 Å;
an LPA₂ Agonist (A) has a distance between A and B of 6-8 Å, a distance between B and C of 15.5-17.5 Å, and a distance between A and C of 18.5-20.5 Å;
an LPA₂ Agonist (B) has a distance between A and B of 10-12 Å, a distance between B and C of 12-14 Å, and a distance between A and C of 18.5-20.5 Å;
an LPA₃ Antagonist has a distance between A and B of 8-14 Å, a distance between B and C of 7-12 Å, and a distance between A and C of 12-16 Å;
an LPA₃ Agonist has a distance between A and B of 8.6-10 Å, a distance between B and C of 4.8-5, and a distance between A and C of 13.4-14.8;
anionic functional groups comprise either phosphate, carboxylate, sulfate, sulfonamide, sulfite, nitro, tetrazole, phosphonamide, amide, hydroxy-oxazole, hydroxyl-thiazole, or combinations thereof; and
hydrophobic functional groups comprise saturated or unsaturated aliphatic, aromatic alkyl, or heteroaromatic alkyl.

3. A method as in claim 1 wherein the S1P₁₋₅ receptor ligand pharmacophore comprises pharmacophore features of Scheme 2 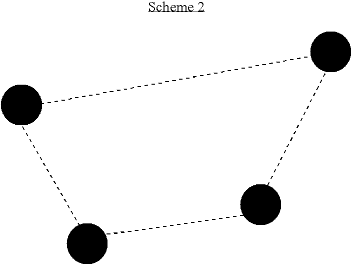
where:
A is an anionic functional group;
B is a cationic or hydrophobic functional group;
C and D are hydrophobic functional groups;
an S1P₁ Agonist has a distance between A and B of 5-7 Å, a distance between A and C of 10.5-11.8 Å, a distance between A and D of 13-16 Å, a distance between B and C of 5.5-7 Å, a distance between B and D of 9-9.5 Å, a distance between C and D of 4.5-5.5 Å, and B is a hydrophobic functional group;
an S1P₂ Agonist has a distance between A and B of 3-5.7 Å, a distance between A and C of 7.5-9.0 Å, a distance between A and D of 14.9-17.3 Å, a distance between B and C of 3.0-6.9 Å, a distance between B and D of 12.4-16.1 Å, and a distance between C and D of 10.3-12.0 Å;
an S1P₃ Antagonist has a distance between A and B of 2.4-3.3 Å, a distance between A and D of 6.1-8.4 Å, a distance between B and C of 2.4-6.1 Å, and a distance between C and D of 5.1-7.9 Å;
an S1P₄ Agonist has a distance between A and B of 3-4 Å, a distance between A and C of 9-10 Å, a distance between A and D of 17-20 Å, a distance between B and C of 9-10 Å, a distance between B and D of 16.5-18.5 Å, and a distance between C and D of 9-10 Å;
anionic functional groups comprise phosphate, carboxylate, sulfate, sulfonamide, sulfite, nitro, tetrazole, phosphonamide, amide, hydroxy-oxazole, hydroxyl-thiazole and trifluoromethyl;
hydrophobic functional groups comprise saturated or unsaturated aliphatic, aromatic alkyl or heteroaromatic alkyl; and
cationic functional groups comprise amine and guanidine functional groups optionally substituted by aromatic hydrogens on electron-deficient aromatic systems (i.e., those with nitro, trifluoromethyl and related substituents).

4. A method of producing an LPA₁-specific response in a human or animal subject, the method comprising administering a composition comprising one or more LPA₁ receptor antagonists as in formula I 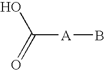
where
B is substituted or unsubstituted aromatic or heteroaromatic;
A is either a direct link, C₀₋₅ substituted or unsubstituted alkyl, 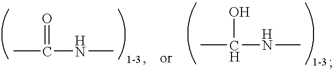
and wherein the LPA₁ receptor antagonist comprises the pharmacophore features of Scheme I 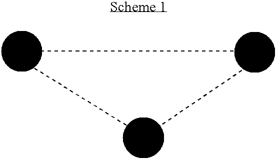
wherein
A is an anionic functional group;
B and C are hydrophobic functional groups; and
the distance between A and B is 7-11 Å, the distance between B and C is 5-10 Å, and the distance between A and C of 6-12 Å.

5. The method of claim 4 wherein the distance between A and B is 7-11 Å, the distance between B and C is 5-8 Å, and the distance between A and C is 6-12 Å in the one or more LPA₁ receptor antagonists described by Scheme I.

6. The method of claim 4 wherein the distance between A and B is 7-11 Å, the distance between B and C is 6-10 Å, and the distance between A and C is 8-12 Å in the one or more LPA₁ receptor antagonists described by Scheme I.

7. An LPA receptor ligand comprising at least one anionic functional group and substituted or unsubstituted aromatic or heteroaromatic alkyl, the LPA receptor ligand being described by 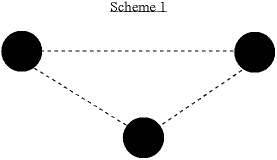
where
A is an anionic functional group;
B and C are hydrophobic functional groups;
an LPA₁ Antagonist (A) has a distance between A and B of 7-11 Å, a distance between B and C of 6-10 Å, and a distance between A and C of 8-12 Å;
an LPA₁ Antagonist (B) has a distance between A and B of 7-11 Å, a distance between B and C of 5-8 Å, and a distance between A and C of 6-12 Å;
an LPA₁ Agonist has a distance between A and B of 15-17 Å, a distance between B and C of 9.2-11.2 Å, a distance between A and C of 15.5-17.5 Å;
an LPA₂ Antagonist has a distance between A and B of 5-9 Å, a distance between B and C of 4-7 Å, and a distance between A and C of 4-6 Å;
an LPA₂ Agonist (A) has a distance between A and B of 6-8 Å, a distance between B and C of 15.5-17.5 Å, and a distance between A and C of 18.5-20.5 Å;
an LPA₂ Agonist (B) has a distance between A and B of 10-12 Å, a distance between B and C of 12-14 Å, and a distance between A and C of 18.5-20.5 Å;
an LPA₃ Antagonist has a distance between A and B of 8-14 Å, a distance between B and C of 7-12 Å, and a distance between A and C of 12-16 Å;
an LPA₃ Agonist has a distance between A and B of 8.6-10 Å, a distance between B and C of 4.8-5, and a distance between A and C of 13.4-14.8;
anionic functional groups comprise either phosphate, carboxylate, sulfate, sulfonamide, sulfite, nitro, tetrazole, phosphonamide, amide, hydroxy-oxazole, hydroxyl-thiazole, or combinations thereof; and
hydrophobic functional groups comprise aromatic alkyl or heteroaromatic alkyl.

8. A composition as in claim 7 wherein the at least one anionic functional group comprises phosphate, carboxylate, or sulfate.

9. A composition as in claim 7 wherein the at least one anionic functional group is linked to the substituted or unsubstituted aromatic or heteroaromatic alkyl by a direct link.

10. A composition as in claim 7 wherein the at least one anionic functional group is linked to the substituted or unsubstituted aromatic or heteroaromatic alkyl by C₀₋₅ substituted or unsubstituted alkyl, [chemical structure]
